(19) United States
(12) Patent Application Publication
(10) Pub. No.: US 2025/0215871 A1
(43) Pub. Date: Jul. 3, 2025

(54) LINEAR PERISTALTIC PUMP

(71) Applicant: Cellular Origins Limited, Melbourn (GB)

(72) Inventors: Daniel Strange, Melbourn (GB); Peter Crossley, Melbourn (GB); Edwin Stone, Melbourn (GB); Adrian Woodward, Melbourn (GB); Leo Steenson, Melbourn (GB)

(73) Assignee: Cellular Origins Limited, Melbourn (GB)

(21) Appl. No.: 18/852,208

(22) PCT Filed: Mar. 31, 2023
(86) PCT No.: PCT/GB2023/050866

§ 371 Date: Sep. 27, 2024

(30) Foreign Application Priority Data
Mar. 31, 2022 (GB) 2204726.0
Jan. 19, 2023 (GB) 2300830.3

(51) Int. Cl. F04B 43/12; F04B 51/00

(52) U.S. Cl. CPC F04B 43/1223; F04B 51/00

(57) ABSTRACT

A linear peristaltic pump (200) for a bioprocessing system comprises: at least one pair of jaws formed by a first jaw (201) and a second jaw (202), the first jaw (201) being moveable relative to the second jaw (202) such that the pair of jaws can be moved between an open configuration in which the jaws are spaced apart for receiving a flexible tube (155) therebetween and a closed configuration in which the jaws are brought together so as to retain the tube therebetween; an actuator mechanism configured to control movement of the first jaw relative to the second jaw; and means (220a-220m) for effecting a peristaltic pumping action on a fluid contained within the tube (155) when the tube is retained between the pair of jaws in the closed configuration.

### FIELD OF THE INVENTION

The present invention relates to a peristaltic pump, which may be used in an automated bioprocessing system to perform automated cell therapy, for example.

### BACKGROUND

Therapeutics are increasingly using cells rather than small molecules as the starting point. The approaches to manufacturing these products are rapidly evolving to keep up with constantly emerging new therapies. In recent years, there has been an increased use of a number of new classes of cell therapies. One class is autologous cell therapies.

Autologous cell therapies are a promising class of therapy, which have significant clinical and commercial potential ranging from treating cancer to fixing genetic defects. These therapies involve taking cells from a patient, manipulating the cells over the course of days to weeks, and re-introducing the cells back into that patient's body to produce a therapeutic effect. The steps taken during autologous cell therapies are often complex; for example a typical CAR-T process may involve a sequence of steps starting with a cryopreserved leukopak, thawing, washing to remove DMSO, enrichment of T cells, activation, transduction, expansion, concentration, formulation fill finish into an IV bag, and cryopreservation, with several other intermediate washing steps. To date, these processes have typically been performed with labour intensive manual processes. In order to perform such processes, fluid such as reagents and patient samples may need to be transferred between large numbers of separate containers, which may be linked together with flexible tubes.

Due to the complexity of bioprocessing, there is a desire to automate the (cell therapy) process while maintaining a closed system that removes the need to perform the steps in a high grade cleanroom. A closed system is one where there is no exposure of the process to the surrounding environment such that there can be no ingress of contaminants from the environment or cross contamination from other processes that are being performed simultaneously.

One way to transfer fluid between the containers while maintaining a closed system is to use a peristaltic pump suitable for use with a disposable or replaceable flexible tube. Peristaltic pumps operate by compressing a flexible tube, and subsequently moving the compressed portion of the tube along its length thereby forcing the fluid through the tube. This may be performed in a number of ways. Rotary pumps, which are commonly used in bioprocessing, utilize a revolving set of rollers, which roll along a loop of the tube during the rotation, thereby compressing the tube and forcing the liquid around the loop. Alternatively, linear peristaltic pumps, which have largely been confined to use in infusion systems and intravenous pumps rather than bioprocessing, utilize a number of individual pressing elements that compress the tubing against a reaction plate in different locations along a linear axis. By coordinating the motion of the individual pressing elements, the compressed portion of the tube may be moved along its length, thereby forcing the fluid through the tube.

However, existing peristaltic pumps have some disadvantages. If the pump is to work effectively, the flexible tubes must be correctly located within the pump, requiring precise manual manipulation from an operator. For example, rotary peristaltic pumps require a complicated installation to insert, bend, and wrap the tube around the pump by an operator. To overcome this challenge, some rotary peristaltic pumps have been designed with snap-fit cartridges that pre-bend the tube, but this still requires preinstalling a connector onto the tubing. While linear peristaltic pumps may have a simpler installation process, existing pumps still require manual installation and alignment of the tube into the pump, and manual closing of the reaction plate against the pressing elements, and typically also have pre-installed mounts on the tubing to aid with this installation.

Furthermore, rotary peristaltic pumps may lead to excessive wear of the tubes. Due to friction in the roller bearings of a rotary pump, a cyclical shear force is applied tangential to the tube. Over time, this may cause the tube to fail through longitudinal creases, transverse tear, and scalloped shaped defects. This means that rotary peristaltic pumps are only suitable for use with a limited number of materials.

Therefore, there is a need for a reliable way to install flexible tubes within a peristaltic pump without the need for precise manipulation of the tube and the pump by an operator. In particular, there is a need for a pump that can automatically interface with a tube without human interaction. In this way, by automating the installation of the flexible tube within the peristaltic pump, a bioprocessing method may be performed autonomously without need for any operator intervention.

### SUMMARY OF INVENTION

Disclosed herein is a linear peristaltic pump for a bioprocessing system, comprising: at least one pair of jaws formed by a first jaw and a second jaw, the first jaw being moveable relative to the second jaw such that the pair of jaws can be moved between an open configuration in which the jaws are spaced apart for receiving a flexible tube therebetween and a closed configuration in which the jaws are brought together so as to retain the tube therebetween; an actuator mechanism configured to control movement of the first jaw relative to the second jaw; and means for effecting a peristaltic pumping action on a fluid contained within the tube when the tube is retained between the pair of jaws in the closed configuration.

By providing an actuator mechanism to control movement of the first jaw relative to the second jaw, the tube may retained within the pump without the need for an operator. Either or both of the first jaw and the second jaw may be moved by the actuator mechanism in order to move the pair of jaws between the open and the closed configuration. The actuator mechanism allows for movement of the jaws without them needing to be moved manually by a human operator. The actuator mechanism may comprise a motor (e.g. a servo motor), a linear actuator, a piston, a camming mechanism, or any other appropriate way (e.g. means, device or mechanism) to automate movement of the jaws.

As used herein, the term “bioprocessing” preferably includes cell therapy, such as autologous and allogenic cell therapies, as well as vaccines and (small batch) bioprocess, for example.

As used herein, the term “linear peristaltic pump” refers to a pump where the tube is arranged along a substantially linear axis during a pumping operation. Thus, as will be well understood, the term “linear peristaltic pump” equivalently refers to a peristaltic pump that is configured as a linear peristaltic pump.

The means for effecting a peristaltic pumping action may comprise: a support portion configured to support the tube; and a plurality of pressing elements, each pressing element being moveable relative to the support portion between a first position, in which the pressing element compresses a portion of tube supported by the support member whereby to restrict the flow of fluid through said portion, and a second position, in which said portion of tube is not substantially compressed and the flow of fluid is not restricted.

Advantageously, in a linear peristaltic pump the pressing elements press against the tube in a perpendicular direction, so shearing forces are reduced. This increases the lifetime of the tube, and may allow for materials such as PVC to be used in the peristaltic pump. This is preferred over rotary peristaltic pumps, where the shear forces due to the rollers damage the tubes over prolonged usage. Due to this, PVC tubing (which is usually used for consumables in bioprocessing since it may be welded) is unsuitable for use with rotary peristaltic pumps. To address this, typically the tubes are instead manufactured with specific sections made from an alternate wear-resistant material (such as silicone or Bioprene®), which are then carefully loaded by a user to align the sections of alternative material with the peristaltic pump. This leads to increased complexity of the consumables and tubes, and increased manual intervention required to correctly operate the pump.

Each of the plurality of pressing elements may be moved between the first position and the second position at different times to adjacent pressing elements so as to effect a peristaltic pumping action on the tube. In other words, the pressing elements may be moved “sequentially” where each pressing element may be moved out of phase with adjacent pressing elements, such that the compressed portion of the tube moves along the length of the tube in the direction of pumping. This motion of the pressing elements may be referred to as a “compression wave”.

Preferably, at least one of the pressing elements is configured always to be in contact with a tube retained between the jaws when in the closed configuration. In this way, the tube is always engaged by the jaws in the closed configuration to prevent the tube from falling out of the pump.

The peristaltic pump may further comprise a drive mechanism for moving the pressing elements individually relative to the support portion so as to effect a peristaltic pumping action on a fluid contained within a tube retained between the jaws. The term “individually” preferably indicates that the pressing elements may move out of phase with each other, such as according to the sequential motion described above. The motion of adjacent pressing elements may be correlated.

The drive mechanism may comprise a camshaft having one or more cams and/or defining a cam profile thereon, and each of the pressing elements comprises a cam contact portion arranged to be in sliding contact with one of said cams and/or said cam profile, whereby each of the individual pressing elements is moved between its first and second positions in a predetermined sequence, and preferably a linear sequence, by rotation of said camshaft.

The drive mechanism may comprise an electric motor, preferably configured to rotate the camshaft at a constant speed during a pumping operation. In this way, a constant flow rate and pressure of fluid may be achieved, and pulsatile flow may be minimised.

The drive mechanism may comprise a plurality of actuators arranged to drive each of the plurality of pressing elements independently. The term “independently” preferably connotes that each pressing element may be moved without requiring movement of any of the remaining pressing elements. The plurality of actuators may also be used to provide the actuation mechanism for movement of the jaws between the open and closed configuration.

The drive mechanism may be configured to provide a particular pumping (or “displacement”) profile. For example, where the drive mechanism comprises one or more cams, the shape of the cams may be adjusted in order to provide the pumping profile. The cams may be arranged to define an asymmetrical profile, e.g., where the pressing elements initially compress the tube quickly (e.g. with a relatively steeper gradient or a greater angle formed by two or more adjacent pressing elements towards an upstream end of the portion of tube), before gradually (i.e. more slowly) releasing the tube (e.g. with a relatively shallower gradient or smaller angle formed by two or more adjacent pressing elements towards a downstream end of the portion of tube). The gradients and/or angles here are described relative to each other, e.g. defined by relative movement of the pressing elements upstream vs. downstream of a portion of (e.g. an occlusion in) the tube. In this way, the piston displacement profiles may be configured as asymmetric profiles to minimise any tangential rubbing that could occur between the pressing elements and tube as the tube deforms through the cyclical process. In other words, the pressing elements upstream of an occlusion in the tube may be designed to compress the tube more steeply, while the pressing elements downstream of the occlusion may be designed to release the tube more shallowly to allow the tube to recover its shape.

Where the drive mechanism comprises a plurality of actuators arranged to drive each of the plurality of pressing elements independently, the actuators may be programmed or otherwise controlled to move at different speeds or in different sequences in order to provide the desired “asymmetrical” pumping profile. By using different pumping profiles, the wear to the tube may be reduced.

Preferably, the peristaltic pump further comprises a detector to determine whether the tube is received between the jaws. The detector may comprise any combination of; a machine vision system, a piezoelectric sensor, a force sensor, and/or a capacitive sensor. A detector for detecting the presence (or absence) of a tube between the jaws may be integrated into at least one or both of the jaws. The detector may comprise a piezoelectric sensor that could also be used to measure the force being applied by the jaws during pumping, and consequently a pressure of a fluid being pumped. Alternatively, or additionally, the detector may comprise one or more capacitive sensors could be used, which could also be used to determine whether the tube contains fluid or air.

Preferably, the actuator mechanism is configured to move the jaws into the closed configuration when the detector determines that the tube is received between the jaws. This reduces the level of operator intervention required in order to operate the pump.

The detector may determine properties of the tube when it is first loaded into the peristaltic pump, such as the outside diameter (OD) of a tube. This may be achieved by detecting a point of contact when the jaws are closed, or by visual inspection (e.g., using a machine vision system) of the diameter or by scanning an identification mark (e.g., visual identification of a label on the tube). Since different tube sizes may be used within a single peristaltic pump (e.g., 4 mm PVC or 6.3 mm C Flex), having automatic detection of the outside diameter reduces the number of manual steps required by the user. The peristaltic pump may be configured to move the jaws and/or pressing elements in response to a measurement by the detector, such as to adjust the distance between the jaws (using the actuator mechanism) so that the distance between the second position of the pressing elements and the support portion corresponds to the outside diameter of the tube.

Alternatively, or additionally, the detector may be configured to determine when the tube is fully occluded when compressed between the jaws and/or by the pressing elements. For example, a force sensor of the detector may provide a force displacement curve as the tube is compressed, with a steeper section of the curve, or a threshold force value indicating that the tube is fully occluded. For example, the transition will be much steeper in the tube if the jaws are designed to fully encase the tubing (such that it is volumetrically locked once it reaches the fully occluded point). Alternatively, a visual sensor may detect when the tube is fully occluded. In this way, the peristaltic pump may be prevented from compressing the tube beyond the fully occluded position. This may be achieved by changing the distance between the jaws, or by otherwise altering the range of motion of the pressing elements (e.g., changing the first position of each of the pressing elements). By preventing “over occlusion” of the tubes, the lifetime of the tubes is increased, and efficiency of the peristaltic pump is improved without affecting performance.

Alternatively, or additionally, the jaws and/or each of the pressing elements may have a corresponding resilient member (e.g., a spring) that limits that maximum force that may be applied to the tube. For example, the actuators of the drive mechanism may have a spring that deforms when a large force is applied, thereby limiting the maximum force that is transmitted to the tube.

The peristaltic pump may further comprise a main body that houses the drive mechanism and/or the actuator mechanism, wherein said pair of jaws extends beyond or outside the main body. The main body may house other components of the pump. In this way, the tube may be located between the jaws without being obstructed by the main body. The tube may be located between the jaws either by manipulating the tube, or by moving the pump so that the tube is located between the jaws.

At least one of the first jaw and the second jaw may comprise a profiled portion, such that when the pair of jaws is moved into the closed configuration the flexible tube is urged towards a predetermined position within the profiled portion. The predetermined position may be a predetermined axis, where the profiled portion aligns the tube along the predetermined axis. The profiled portion may also prevent the tube from being forced out from between the jaws during a pumping operation.

The profiled portion of the jaw may comprise a curved portion of the jaw, preferably having a radius that generally corresponds to the radius of the tube to be received thereby.

The plurality of pressing elements and/or the support portion may be formed from a low friction material to allow for sliding of the tube during a pumping operation.

In this way, the pressing elements only apply a cyclical compressive force, while reducing any cyclical shear forces that may increase wear of the tube. The low friction material may have a coefficient of friction less than 0.5, preferably less than 0.2, such as about 0.1. The low friction material may comprise PTFE.

The peristaltic pump may further comprise one or more gripping mechanisms arranged to engage the tube. The one or more gripping mechanism may be configured to load and/or unload the tube from the pair of jaws when the jaws are in the open configuration. The gripping mechanism(s) preferably remain static once the tube is gripped, preferably during a pumping operation. The gripping mechanism(s) may apply a tensile force to the tube when loading the tube into the jaws (e.g., to pre-tension the tube slightly). This helps to locate the tube in a predetermined position between the jaws and reduces buckling of the tube.

The one or more gripping mechanisms may be configured to retain the tube so as to inhibit longitudinal motion of the tube through the pair of jaws during a pumping operation. In other words, the one or more gripping mechanisms secure the tube at a predetermined position during use of the peristaltic pump (i.e. when the pair of jaws are in the closed configuration). The gripping mechanism may have a first part and a second part configured to secure the tube therebetween. The gripping mechanism may comprise a pair of jaws and/or one or more opposing pairs of pressing elements acting as “gripping elements”. The gripping elements may be provided by one or more of the plurality of pressing elements mentioned above, or the gripping mechanism may be provided separately to the pair of jaws. The gripping mechanism (or the gripping elements) may comprise a high friction material in order to inhibit longitudinal motion of the tube during use of the peristaltic pump. The high friction material may have a coefficient of friction greater than 0.5, preferably more than 0.8, such as about 0.9 or 1. The high friction material may comprise rubber. The gripping mechanism (or the gripping elements) may be positioned at the ends of the peristaltic pump, or adjacent the jaws of the peristaltic pump. Preferably, a pair (two) of gripping mechanisms are provided, with one positioned on either side of the jaws (along the axis of the tube). Preferably, the gripping mechanisms are spaced at least three tube diameters from the endmost pressing elements, in order to allow the tube to recover to its original shape (e.g. so that deformation of the tube by the pressing elements does not interact with the gripping mechanisms).

The gripping mechanism may be provided by one or more of the pressing elements mentioned above. Accordingly, it may be desirable to have a combination of pressing elements with high friction and low friction surfaces, for example having high-friction pressing elements (acting as gripping elements) at opposing ends of the pump, which lightly grip the tube and remain static during a pumping operation, and low-friction pressing elements that compress and occlude the tube, thereby providing a pumping action. In this way, a fixed tangential force can be applied to the tube to restrain it against a pressure wave that is generated by the peristaltic pump, but only apply a cyclical compressive force, thereby avoiding cyclical shear forces that contribute to failure.

Alternatively, the gripping mechanism may be a separate to the pair of jaws containing the pressing elements. For example, the gripping mechanism may be movable independently to the pair of jaws. As used herein, the terms high and low friction material are relative terms, for example comparing rubber and PTFE mentioned above. Preferably, a portion of the gripping mechanism arranged to contact the tube comprises a material with higher friction than a material on a portion of the pair of jaws arranged to contact the tube (e.g., the profiled portion of the support plate and/or the pressing elements).

The gripping mechanism may be movable relative to the jaws in a direction substantially perpendicular to the longitudinal direction (i.e. the longitudinal axis of the tube when retained in the pair of jaws). Before installation of a tube in the peristaltic pump, the gripping mechanism may be aligned with the jaws along a common axis to allow for easy loading of the tube in the peristaltic pump. Subsequently, the gripping mechanism may be moved perpendicular to the axis of the tube, thereby bending a portion of the tube into an S-bend. Alternatively, the gripping elements within the pump may retain the tube in such a way that it is “kinked” relative to a main pumping section formed by the remaining pressing elements. This better retains the tube inside the peristaltic pump and reduces longitudinal motion of the tube through the jaws.

Where longitudinal motion (or “drift”) of the tube may occur through the pump, the peristaltic pump may be configured periodically to pause pumping, release the tube, re-engage it at a different position (e.g. further downstream) and move it back to its original position, before continuing pumping, in order to compensate for the longitudinal motion. For example, this periodic repositioning may occur every 100 cycles. This may allow for all of the points of contact with the tube (including the pressing elements) to comprise a low friction material, thereby further reducing shearing forces.

Alternatively, or additionally, one part (e.g. one side of the pair of jaws) of the gripping mechanism may be provided by the support portion, with the other part of the gripping mechanism pressing against the support portion. For example, the gripping mechanism may be provided by one or more of the pressing elements acting as gripping elements, as mentioned above. In this instance, the support portion may have a high friction material, with the pressing elements having the low friction material; this keeps the tube in place while minimising cyclical shear forces being applied.

The support portion may be arranged to define the first jaw, and the plurality of pressing elements may be arranged to define the second jaw.

The peristaltic pump may further comprise at least one tube retaining element provided on the second jaw for retaining a tube in a predetermined position when the jaws are moved to their closed configuration, preferably wherein the tube retaining element is arranged to mate with the support portion when the tube is retained in the closed configuration. The at least one tube retaining element may also be used to prevent longitudinal motion of the tube during use; therefore the at least one tube retaining element may comprise a high friction material.

The support portion may be defined by a further plurality of pressing elements movable to oppose said plurality of pressing elements of the second jaw.

The peristaltic pump may further comprise a flow sensor configured to measure the flow of fluid through a tube secured between the jaws.

Preferably, the flow sensor is a non-contact flow sensor, for example an ultrasonic flow sensor.

The peristaltic pump may further comprise a pressure sensor configured to measure the pressure of fluid in a tube secured between the jaws.

Preferably, the flow sensor and/or the pressure sensor are arranged to feedback a control signal to the drive dependent on the measured flow or pressure of fluid. In this way, the drive mechanism may be controlled to counteract any flow or pressure pulsations.

The means for effecting a peristaltic pumping action may be configured to effect an asymmetric peristaltic pumping action along a length of the tube. Advantageously, this may reduce wear on the tube. As used herein, the term “asymmetric” peristaltic pumping action preferably connotes that the motion of the pressing elements upstream of the occluded portion of the tube is different to motion of the pressing elements downstream of the occluded portion of the tube. For example, the peristaltic pumping action may be configured to pinch a portion of the tube more quickly than it releases said pinched portion, or vice versa.

Where the drive mechanism comprises one or more cams, this may be achieved by having cams with an asymmetric shape. Where the drive mechanism comprises a plurality of actuators for each of the pressing elements, the actuators may be programmed or controlled to move with specific timings and/or speeds.

Preferably control of the peristaltic pump is automated.

Preferably, the actuator mechanism is configured to receive signals from a controller to move the jaws between the open and closed configurations, preferably wherein the controller is further configured to control the means for effecting a peristaltic pumping action to commence pumping once the jaws are in the closed configuration.

The peristaltic pump may be operated as a “flexible” pinch valve, such as by moving the jaws to the closed configuration, or by moving one or more of the pressing elements to the first position, to pinch a portion of the tube closed.

Also disclosed herein is a robotic device comprising the linear peristaltic pump as described above, preferably wherein the pump forms part of a robotic end effector. In this way, the pump may be moved by the robotic device to locate the tube between the jaws. Optionally, the power supply for the drive mechanism and/or the actuator mechanism may be located external to the peristaltic pump, such as elsewhere in the robotic device. In this way, the size and weight of the end effector is reduced.

Also disclosed herein is a bioprocessing system comprising the linear peristaltic pump or the robotic device described above. Preferably, the bioprocessing system comprises an automated system.

Also disclosed herein is a method of performing a peristaltic pumping action on a tube, comprising: actuating a plurality of pressing elements individually to compress and release a portion of said tube so as to pump a fluid therethrough, wherein said plurality of pressing elements are arranged in a substantially linear configuration.

The compression of said portion of the tube may be referred to as “pinching” or “occluding” said portion of tube. Said portion of tube may comprise a length of the tube, with each of said plurality of pressing elements arranged to engage a different part of said tube. The pressing elements may be actuated individually and/or independently, or in combination. The pressing elements are preferably actuated in a predetermined sequence and/or a series of movements. Actuation of the pressing elements is preferably automated.

Preferably, the plurality of pressing elements engage the tube in a direction that is substantially perpendicular to the surface of said portion of tube (i.e. perpendicular to the longitudinal axis or tangential direction of the tube). Said plurality of pressing elements are preferably arranged adjacently, e.g., each pressing element is adjacent at least one other pressing element, and in some (e.g., most) cases may have another pressing element adjacent on either side of it such that it is sandwiched between two other pressing elements. The tube is preferably retained between a pair of jaws during the peristaltic pumping action.

As used herein, the term “automated system” preferably connotes a system operated and/or controlled by automation, and which term preferably includes one more of the following: robotic devices, conveyers, one or more actuators configured to engage and/or move containers or indeed any combination of these features that are capable of moving and/or manipulating the containers and/or tubes within the system.

As used herein, the term “robotic device” preferably connotes an automated machine or device programmed to perform specific mechanical functions, and which term preferably includes robots, collaborative robots (cobots), x-y or Cartesian-robots, robotic arms, and one or more actuators, possibly also comprising one or more robot end effectors, and will typically also include one or more sensors, microprocessors and power supply. The term “end effector” refers to a tool or manipulator which may form part of any type of “robotic device”, for example as described above.

As used herein, the term “fluid” preferably connotes liquid and/or gas, and may further include material such as cell material contained therein.

It will be understood by a skilled person that any apparatus feature described herein may be provided as a method feature, and vice versa. It will also be understood that particular combinations of the various features described and defined in any aspects described herein can be implemented and/or supplied and/or used independently.

Moreover, it will be understood that the present invention is described herein purely by way of example, and modifications of detail can be made within the scope of the invention. Furthermore, as used herein, any “means plus function” features may be expressed alternatively in terms of their corresponding structure.

### BRIEF DESCRIPTION OF DRAWINGS

An embodiment of the present invention will now be described in detail, purely by way of example, with reference to the accompanying drawings, in which:

FIG. 1 is a schematic diagram of an automated bioprocessing system;

FIG. 2 shows a robotic device having an end effector configured as a linear peristaltic pump;

FIGS. 3a to 3c are schematic diagrams showing cross-sectional side views of a peristaltic pump having a plurality pressing elements prior to a pumping operation, and at two different positions during a pumping operation;

FIG. 4a is a schematic diagram showing a cross-sectional view of the peristaltic pump of FIG. 3 having a pair of gripping elements arranged to retain a portion of tube separate to the pressing elements at either end of a portion of tube;

FIG. 4b shows the peristaltic pump of FIG. 4a with a pair of gripping elements arranged at either end of a portion of tube that are offset perpendicular to a longitudinal axis of the tube;

FIG. 5 is a schematic diagram of a peristaltic pump having a sensor;

FIGS. 6a and 6b are schematic diagrams showing cross-sectional side views of a peristaltic pump in which the pressing elements are individually actuated to compress a portion of the tube;

FIGS. 7a and 7b show perspective and side views, respectively, of an embodiment of a peristaltic pump;

FIG. 7c shows part of the peristaltic pump of FIGS. 7a and 7b;

FIGS. 7d and 7e show schematic diagrams of a cross-sectional end view through the part of the peristaltic pump shown in FIG. 7c;

FIGS. 8a and 8b are end views of the peristaltic pump of FIG. 7 illustrating jaws that move between an open and a closed configuration to receive and retain, respectively, a portion of tube therebetween;

FIGS. 8c to 8e are perspective and sectional views of the support plate of the peristaltic pump of FIG. 7;

FIGS. 9a to 9c are end views of the peristaltic pump with some components removed to illustrate a tube being compressed between the jaws;

FIGS. 10a to 10e are perspective and side views of the pressing elements and the support plate having profiled portions;

FIGS. 11a to 11d show perspective and side views of the pump having a tube latch;

FIG. 12 is a schematic diagram showing a cross-sectional view of another embodiment of a peristaltic pump in which there are opposing pressing elements arranged to compress a portion of the tube;

FIGS. 13a to 13c are schematic diagrams showing cross-sectional end views of another embodiment of a peristaltic pump having a plurality of tube-retaining elements; and

FIG. 14 shows an example of a pumping (or “displacement”) profile that may be used during operation of the peristaltic pump.

### DETAILED DESCRIPTION

In the following description and accompanying drawings, corresponding features may preferably be identified using corresponding reference numerals to avoid the need to describe said common features in detail for each and every embodiment.

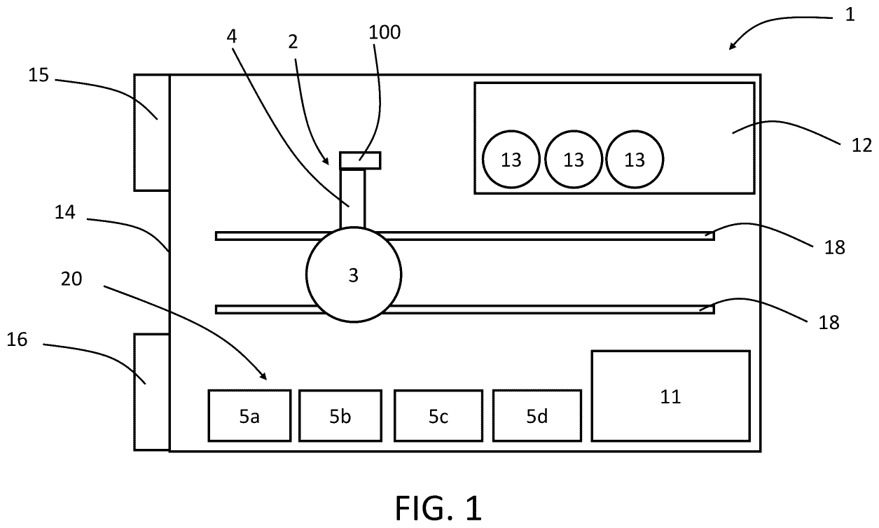

An automated bioprocessing system 1 is shown schematically in FIG. 1. The system 1 has a series (e.g. a “plurality”) of processing stations 20 configured to perform processing steps for bioprocessing, and an (automated) system 2 for automating (at least part of) the process.

In this exemplary system 1, the processing stations 20 include a thawing station 5a, a centrifuge 5b, a magnetic cell separator 5c, a controller rate freezer 5d, and a refrigerator 11, though additional and alternative stations 20 (not shown) for processing can be installed depending on the specific process being performed by the system 1.

The processing stations 20 may include any combination of a concentration station, a cryopreservation unit, a washing station, a cell enrichment station, a cell expansion station, a cell selection station, stations for determining cell count, cell viability or cell type, or stations for any other suitable processing or analysis step. The system 1 also has an incubator 12 that is large enough to contain and incubate multiple consumables 13 at a time, including under perfusion.

For example, the incubator 12 may be capable of storing twenty consumables 13 and operate at around 37° C., though the number of consumables 13 can be chosen to meet the needs of the particular bioprocessing to be performed. Each consumable 13 may contain cellular samples, reagents or fluids, and each consumable 13 connects to a first “upstream” end of a tube (150 not shown) which leads to a second “downstream” end of the tube 150, which is fluidly sealed when unconnected (or “free”). Thus, as referred to herein, a “consumable” may be in the form of a “container”, which may for example hold cell material to be processed in a cell therapy process.

All of the consumables 13 and reagents may be pre-loaded in the system 1 before a particular bioprocess begins, though additional reagents can be added throughout the process if required (for example at day 7 of a 10-day therapy process). The additional reagents may be required for reactivation of cells, or to add additional media to the consumables 13 for example.

A particular bioprocess may be defined by a bioprocessing workflow, and preferably the system 1 can be configured to carry out several bioprocessing workflows. For example, the system 1 can carry out the same bioprocessing workflow in parallel for multiple patient samples, or it can carry out different bioprocessing workflows in parallel for multiple patient samples. Each bioprocessing workflow may use a different subset of the processing stations 20 in the system 1. In a preferred embodiment, the system 1 comprises stations 20 to perform concentrations, washing and incubation processes.

The automated system 2 is configured to install one or more consumables 13 into each of the series of processing stations 20 and to move the consumables 13 between stations 20. The automated system 2 in this exemplary system 1 includes a robotic device 3 that can move the consumables 13 between the various stations 20, and can manipulate the tubes 150 connecting to each of the consumables 13.

The robotic device 3 may be mounted on rails 18, which allows the robotic device 3 to have access to all areas of the system 1 such as the stations 20. The robotic device 3 may be configured as a co-operative robot (“cobot”). The robotic device 3 may be an XY or Cartesian robot, or a robot on a gantry, for example. The robotic device 3 may have a robotic arm 4 for manipulating the consumables 13 and tubes 150, as shown here, or may include a conveyer belt, one or more actuators, or any combination of the above aspects. The robotic device 3 may have an end effector 100 for manipulating and interacting with the tubes 150 and consumables 13. The end effector 100 may be located on the robotic arm 4, or may be located on the XY or Cartesian robot.

The automated system 2 is configured to manipulate a fluid connection between a first consumable 13 and a separable second consumable whereby to create an aseptic connection that enables a controlled transfer of fluid or cell material between the first consumable 13 and the second consumable 13. Here, the robotic device 3 is used to form (or manipulate) fluid connections between the tubes 150 so that separate consumables 13 can be connected together.

The system 1 also has a pumping unit (not shown) which pumps fluid along the tubes 150 once the robotic device 3 has successfully connected two consumables 13 via their respective tubes 150. The pumping unit may configured as the end effector 100 described above, and/or may be located on the robotic arm 4. The pumping unit may be located on a separate robotic arm (not shown). Alternatively, the pumping unit may be a static component placed at one of the stations 20 into which the tubes 150 are placed by the robotic arm 4 for pumping to occur.

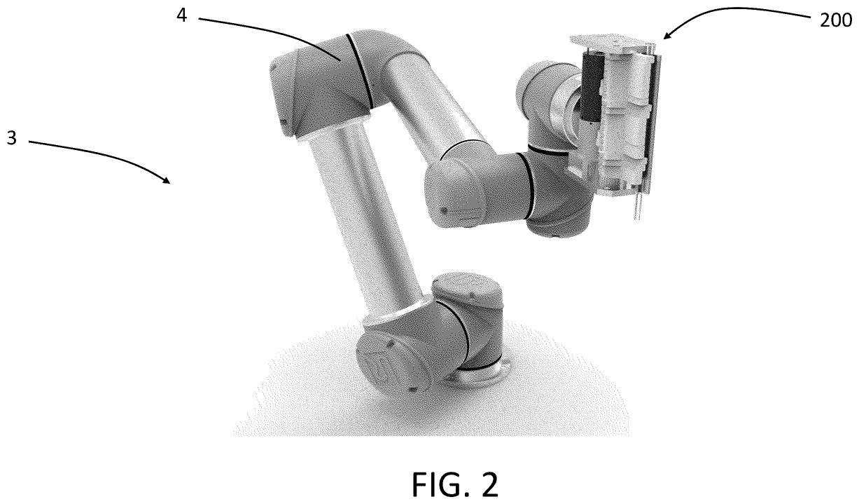

FIG. 2 depicts a robotic device 3, which may form part of the automated system 2 described above. The robotic device 3 comprises a pumping unit in the form of a linear peristaltic pump 200 according to the present invention located on the robotic arm 4. In FIG. 2, the pump 200 is depicted as holding the tube 150 vertically, but the robotic arm 4 may move pump 200 to any other orientation when fluid is pumped through the tube 150. The pump 200 comprises a pair of jaws, moveable between an open position and a closed position for receiving and retaining therebetween a flexible tube, through which a fluid contained therein can be moved by the pump 200. The automated system 2 is configured to locate the tube 150 between the jaws of the peristaltic pump 200, actuate the jaws to close around the tube 150, and then commence pumping.

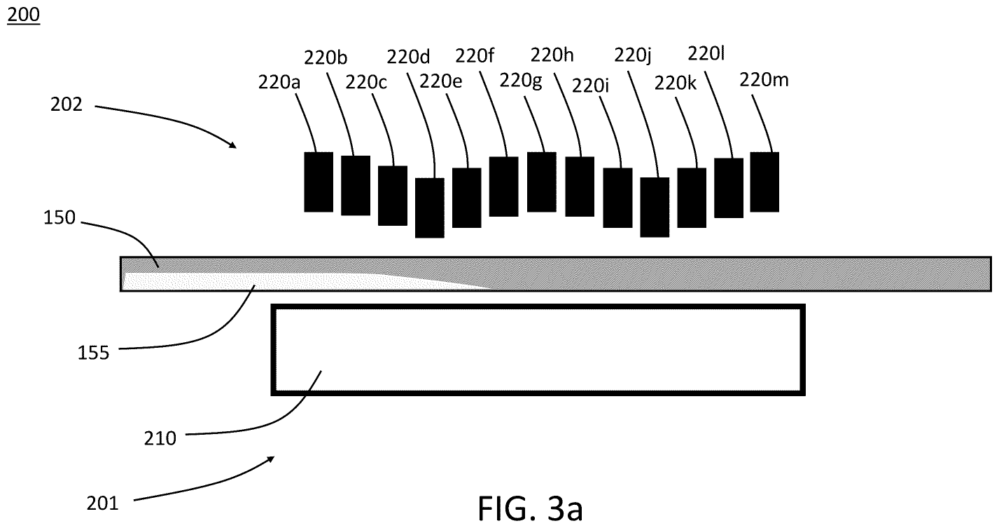

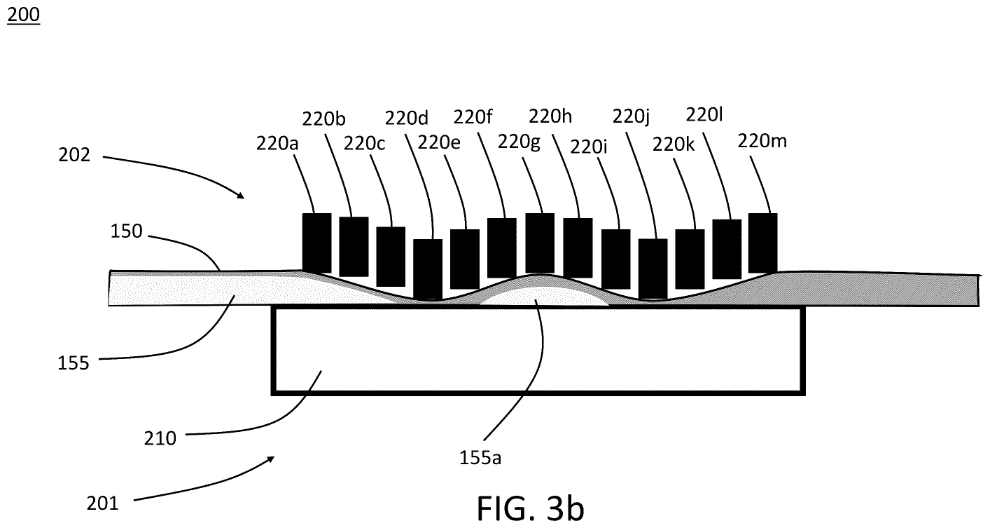

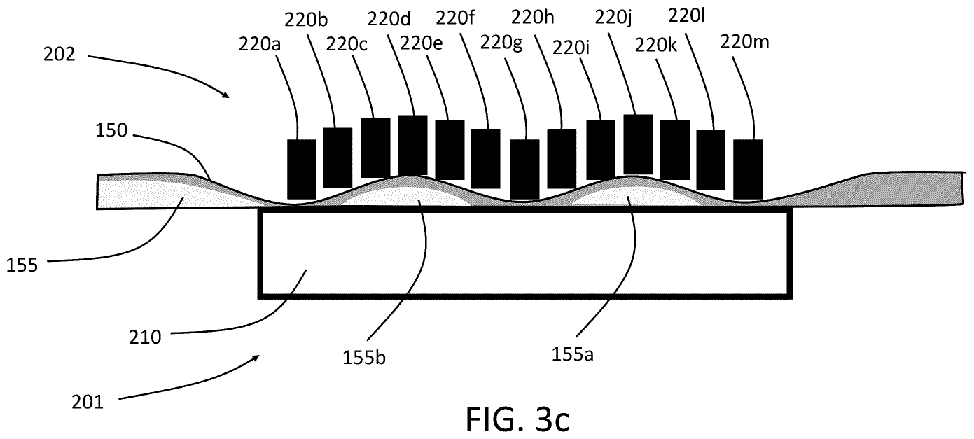

FIG. 3a depicts a simplified cross-sectional side view of a linear peristaltic pump 200 and a tube 150 containing a fluid 155 at an initial time, To, prior to a pumping operation. The pump 200 comprises a “reaction portion” or “support portion”, here in the form of a support plate 210 that provides a first jaw 201, and a plurality (e.g. “array”) of pressing elements 220a-220m that provide a second jaw 202. The first and second jaws 201, 202 thereby together define a pair of jaws. The jaws may together be defined as having a “clam-shell” arrangement. The support plate 210 is referred to as the first jaw 201 in this embodiment simply for convenience. Alternatively, the support plate 210 may provide the second jaw 202, and the plurality of pressing elements 220a-320m may provide the first jaw 201. In this example, thirteen pressing elements 220a-220m are depicted, but more or fewer pressing elements 220 may be used.

At least one of the first jaw 201 and the second jaw 202 may be moved relative to the other of said second jaw 202 or first jaw 201, so that the tube 150 may be received and/or located therebetween. FIG. 3a depicts the pump 200 in an open configuration. In this open configuration, the first jaw 201 is moved away from the second jaw 202 so that the tube 150 may be located between the jaws 201, 202.

Preferably, none of the pressing elements 220 of the second jaw 202 press the tube 150 against the support plate 210 when the jaws 201, 202 are in the open configuration.

FIG. 3b depicts a simplified cross-sectional side view of the pump 200 at a first time, T₁, during a pumping operation. In FIG. 3b, the pump 200 is in a closed configuration whereby the first jaw 201 has been moved towards the second jaw 202, and one or more of the pressing elements 220 is compressing the tube 150. The jaws 201, 202 may be moved between the open and the closed configuration in a number of ways. For example, the first jaw 201 may be moved by an actuator mechanism, thereby moving the first jaw 201 relative to the second jaw 202, or vice versa (or both jaws 201, 202 may be moved). In this way, the tube 150 is retained between the first jaw 201 and the second jaw 202.

Each of the pressing elements 210a-210m are movable between a first position where the pressing element 220 is closest (e.g. proximal) to the support plate 210, and a second position where the pressing element 220 is furthest (e.g. distal) from the support plate 210. As will be described later, the pressing elements 220 are moved by a drive mechanism. When a tube 150 is present in the linear peristaltic pump 200, in the first position the tube 150 is maximally compressed by the pressing element 220, and in the second position the tube 150 is less compressed by the pressing element 220. Preferably, in the first position the tube 150 is pinched shut (e.g. “occluded”) by the pressing element 220, such that movement of fluid 155 is inhibited from on one side of the pressing element 220 to the opposite side of the pressing element 220, and more preferably such that fluid 155 is unable to move along the tube 150 past the pressing element 220. Preferably, in the second position, fluid 155 may move through the tube 150 past the pressing element 220, though the tube 150 may still be (at least partially) compressed by the pressing element 220. As depicted, pressing elements 220d and 220j are in the first position, and pressing elements 220a, 220g, and 220m are in the second position. The remaining pressing elements 220b, 220c, 220e, 220f, 220h, 220i, 220k, and 220l are at a position somewhere between the first position and the second position. The tube 150 contains a fluid 155 which has a first volume 155a that is isolated from other parts of the tube 150 by the pressing elements 220d, 220j.

In order to pump the fluid 155, the pressing elements 220 are each moved between the first position and the second position. The pressing elements 220 are moved smoothly between the first and the second position so that the tube 150 is gradually compressed and partially released by each of the pressing elements 220. The motion of each pressing element 220 occurs sequentially. According to a predetermined sequence, the motion of a particular pressing element 220 is offset relative to the adjacent pressing elements 220 so that the pressing elements 220 reach the first and second position at different times. For example, in order to pump fluid towards the right, following the configuration depicted in FIG. 3b, pressing elements 220e and 220k will be the next ones to reach the first position, and 220b and 220h will be the next ones to reach the second position. As the motion continues, the portion of the tube 150 that is maximally compressed will gradually move along the tube 150 in the direction of pumping. Likewise, the less compressed portion of the tube 150, and any fluid 155 contained therein, also moves along the tube 150 in the direction of pumping. This continued motion of the pressing elements 220 may be referred to as a compression wave.

As such, the arrangement of pressing elements 220 in this embodiment provides a means for effecting a peristaltic pumping action on a fluid contained within the tube 150 when the tube 150 is retained between the pair of jaws 201, 202 in the closed configuration.

FIG. 3c shows a simplified schematic diagram of the pump 200 at a second time, T₂, during the pumping operation that is later than the first time, T₁, shown in FIG. 3b. This position is halfway through the cycle of operation, such that the pressing elements 220a, 220g, 220m have now moved into the first position, and the pressing elements 220d, 220j have moved into the second position. The first volume of fluid 155a has moved further to the right in the direction of pumping, and a second volume of fluid 155b has now become isolated from other parts of the tube 150 by pressing elements 220a, 220g.

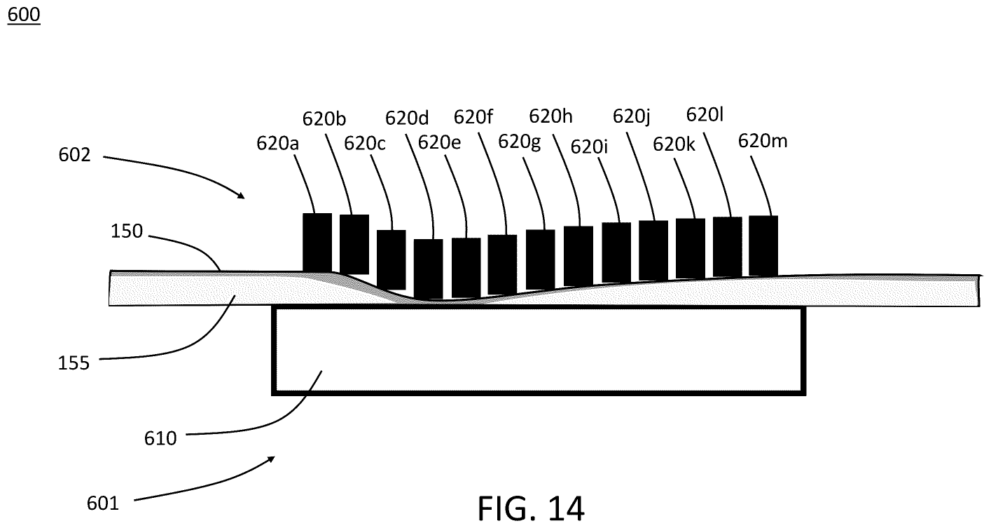

The sequence described above may be continued until the desired volume of fluid 155 has been pumped along the tube 150. The sequence may also be reversed so that fluid 155 may be pumped in the opposite direction along the tube 150. As will be described later, one or both of the jaws 201, 202 may have a profiled portion so that the tube 150 is automatically aligned (e.g. urged towards the profiled portion) between the jaws 201, 202 when the jaws 201, 202 are moved from the open configuration to the closed configuration. As will be described later in relation to FIG. 14, a particular pumping (or “displacement”) profile may be used in order to reduce wear to the tubes 150 and/or increase efficiency of the pump 200.

The pressing elements 220 and/or the support plate 210 may comprise a low friction material on a surface that contacts the tube 150 in order to reduce shear forces applied to the tube 150. In this way, the pressing elements 220 only apply a compressive force to the tube 150 in a direction substantially perpendicular to its surface, without any significant force being applied tangent to its surface. This can reduce wear to the tube 150, thereby increasing its lifetime. The low friction material may have a coefficient of friction less than 0.5, preferably less than 0.2, such as about 0.1. The low friction material may be PTFE (or a PTFE coating), Acetal (either lubricated or unlubricated), polycarbonate, UMWE, PEEK, or any combination of the above.

The pump 200 may comprise a detector to determine whether the tube 150 Is received between the jaws 201, 202. For example, at least one of the jaws 201, 202 may have at least one tube sensor (not shown) to detect the presence of the tube 150. The detector may determine properties of the tube 150 when it is first loaded into the pump 200, such as the outside diameter (OD) of the tube 150. This may be achieved by detecting a point of contact when the jaws 201, 202 are closed. The tube sensor may be a piezoelectric sensor to detect the force applied between the jaws 201, 202 and the tube 150. The force measured by the tube sensor may be used to calculate the pressure inside the tube 150 or to identify the type of tubing (e.g. material properties, wall thickness, and/or outer diameter) from its force deflection characteristics. The tube sensor may be a capacitive sensor, which may determine whether the tube 150 contains a fluid or a gas. Any combination of the above tube sensors may be used on either or both of the jaws 201, 202. Tube sensors may also be provided using a machine vision system or another means for “visual” inspection. The outer diameter may therefore be determined by visual inspection of the diameter or by scanning an identification mark (tubes 150 may be laser marked to provide identification marks). The distance between the jaws 201, 202 may be adjusted based on the outer diameter of the tube 150.

The tube sensor may detect a force displacement curve as the tube 150 is compressed by the jaws 201, 202. A steeper section of the curve, or a force greater than a threshold value may indicate that the tube 150 is fully occluded. Particularly, when the jaws 201, 202 are designed to fully encase the tube 150 (such that it is volumetrically locked once fully occluded), the increase in force may be substantial. The pump 200 may be prevented from compressing the tube 150 beyond this fully occluded position. For example, the distance between the jaws 201, 202 may be adjusted, or the first position of the pressing elements 220 may be adjusted. By preventing over occlusion of the tube 150, wear is reduced and the lifetime of the tube 150 may be increased. Furthermore, by limiting the maximum force that is applied, the efficiency of the pump 200 may be improved without affecting performance. A resilient member (not shown) such as a spring may be provided in the jaws 201, 202 and/or in each of the pressing elements 220 to reduce the maximum force applied to the tube 150.

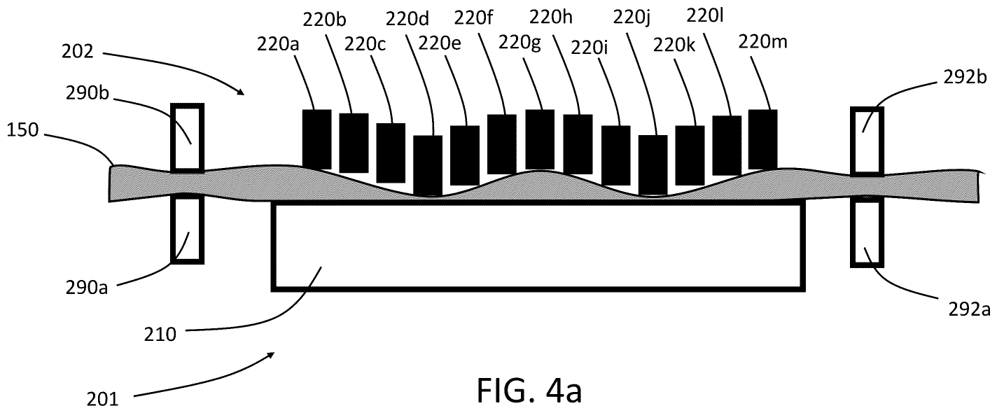

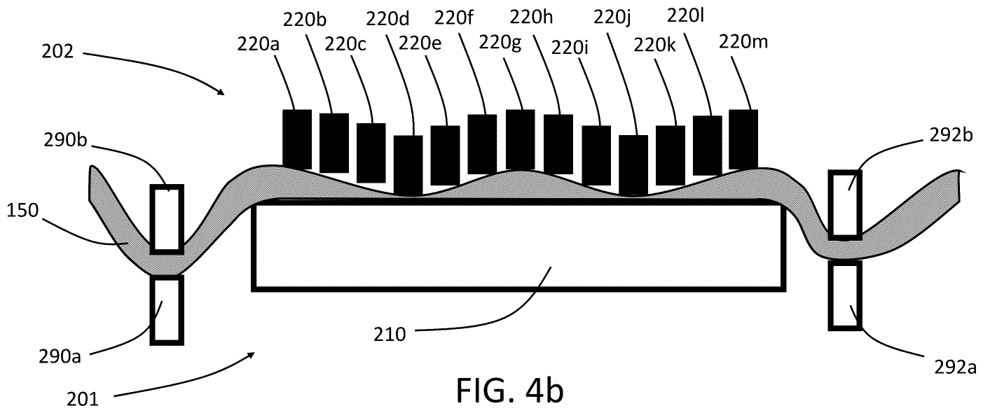

As will now be described in relation to FIG. 4a, the pump 200 may also include a first gripping mechanism 290 and a second gripping mechanism 292 for gripping (and thereby retaining) the tube 150. As shown, the gripping mechanisms 290, 292 are positioned on either side of the jaws 201, 202 (along the axis of the tube 150). Preferably, the gripping mechanisms 290, 292 are spaced at least three tube diameters from the endmost pressing elements 220, so as not to interfere with deformation of the tube 150 and/or recovery of the tube 150 to its original shape. In other words, deformation of the tube 150 by the pressing elements 220 is not interfered with by the gripping mechanisms 290, 292.

Each of the gripping mechanisms 290, 292 are divided into a first part 290a, 292a, that oppose a corresponding second part 290b, 292b, thereby defining a pair of jaws. It will be appreciated that each of the gripping mechanisms 290, 292 may also be provided by a plurality of individual pressing elements (e.g. acting as gripping elements) and/or support plates, in a similar manner to the jaws 201, 202. While two gripping mechanisms 290, 292 are described herein, it will be appreciated that a single gripping mechanism may be used.

The gripping mechanisms 290, 292 may be operated so that a tube 150 can be gripped by the gripping mechanisms 290, 292 and positioned within the pump 200. In order to grip the tube 150 with one or both of the gripping mechanisms 290, 292, the first part 290a, 292a and second part 290b, 292b are moved so that the tube 150 is received between the jaws thereby defined while in an open configuration. Subsequently, the first part 290a, 292a and second part 290b, 292b may be moved towards each other to grip and thereby retain the tube 150 therebetween in a closed configuration. Preferably, the tube 150 is not pinched shut by the gripping mechanisms 290, 292 so that fluid 155 contained in the tube 150 can be pumped past them. During a pumping operation, the gripping mechanisms 290, 292 preferably remain static (i.e. they do not substantially move relative to the jaws 201, 202, and the first part 290a, 292a does not substantially move relative to the second part 290, 292b).

The gripping mechanisms 290, 292 may comprise a high friction material on a surface or tip which contacts the tube 150. In this way, even when low friction materials are used for the pressing elements 220 and/or support plate 210, longitudinal motion (e.g. “wandering”) of the tube 150 through the pump 200 is reduced. The high friction material may have a coefficient of friction greater than 0.5, preferably more than 0.8, such as about 0.9 or 1. The high friction material may comprise rubber (e.g., Butyl, Silicon, EPDM rubbers), TPE, a metal such as steel or aluminium (which may be serrated or profiled), or any combination of the above.

For example, for a relatively high pressure of 1 bar with a PVC tube 150 with inner diameter of 2.4 mm, the tangential force to hold the tube 150 will be on the order of 1 N. For a 6 mm wide pressing element 220, it takes approximately 30 N to fully occlude the tube 150. Therefore, a gripping mechanism (with two 6 mm gripping elements with coefficient of friction of 1) would apply approximately 6 N in total (e.g., 3 N for each gripping element), in order to prevent longitudinal motion of the tube 150. Such a force would only occlude the tube 150 by about 10% which would not adversely affect operation of the pump 200 while still keeping the tube 150 in place.

The gripping mechanisms 290, 292 may also be moved away from each other to stretch the tube 150 longitudinally when in the closed configuration, thereby ensuring it is positioned correctly within the pump 200. In this way, when each of the pressing elements 220 are moved relative to the support plate 210 to compress a portion of the tube 150, the tube 150 will be correctly aligned in the predetermined position. ditionally, the gripping mechanisms 290, 292 may apply a predetermined force when stretching the tube 150, thereby pretensioning the tube 150. In this way, the tube 150 may be pinched more consistently by the pressing elements 220 during a pumping operation.

As shown in FIG. 4b, the gripping mechanisms 290, 292 may be movable relative to the jaws 201, 202 in a direction generally perpendicular to the longitudinal direction (i.e. generally perpendicular to the axis) of the tube 150. The gripping mechanisms 290, 292 may be moved into the position shown in FIG. 4b after installation of the tube 150 in the pump 200. By moving the gripping mechanisms 290, 292 into this “offset” position (relative to the axis of the tube 150) during use of the pump 200, the tube 150 is forced into an S-bend position, which forms a kink in the tube 150 that better retains the tube 150 in the pump 200 and can reduce longitudinal motion of the tube 150 through the jaws 201, 202.

The gripping mechanisms 290, 292 may be located on a separate robotic arm 4 to the pump 300 or may be located on the same robotic arm 4. The gripping mechanisms 290, 292 may also perform other operations in the bioprocessing system 1, such as manipulation of the consumables 13 and the tubes 150 and/or forming the fluid connections between the tubes 150.

Alternatively or additionally, the tube 150 may be allowed to move longitudinally through the pump 200, which may further reduce shear forces applied by holding it in place. However, the jaws 201, 202 may periodically release the tube 150, and re-engage it at a different position (e.g., further downstream) in order to compensate for the gradual longitudinal drift of the tube 150, before moving it back to its original position. For example, this readjustment may occur every 100 cycles. The gripping mechanisms 290, 292 may be used to move the tube 150 in the longitudinal direction when the tube 150 is released by the jaws 201, 202.

As a further use for the pump, the jaws 201, 202, the pressing elements 220 and/or the gripping mechanisms 290, 292 may be used to operate the pump 200 as a pinch valve, which may be useful when the pump 200 is used as part of a bioprocessing system. In particular, the jaws 201, 202 may be moved to the closed configuration, the pressing elements 220 may be moved to their first position, and/or the gripping elements 290, 292 may be moved beyond their closed configuration to pinch the tube 150 shut.

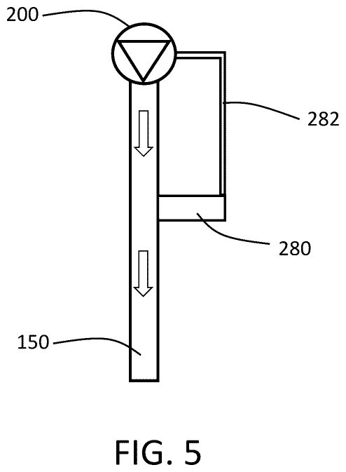

FIG. 5 shows a sensor 280 for the tube 150 located downstream of the pump 200. Alternatively, the sensor 280 may be located upstream of the pump 200, or sensors 280 may be positioned both upstream and downstream of the pump 200. The sensor 280 may be used in addition to, or instead of the one or more tube sensors described above. The sensor 280 is located in a feedback loop 282 to the drive mechanism of the pump 200. The sensor may comprise a flow sensor 280; based on the measured flow through the flow sensor 280, the drive signal to the drive mechanism may be altered to counteract any potential flow pulsations. The flow sensor 280 may help to address or verify volume accuracy of the pump 200 and/or to compensate for variations in mechanical properties of the tube 150 with temperature and rate of operation. The flow sensor 280 may be any suitable flow sensor 280 including a non-contact ultrasonic flow sensor 280, such as a Sonoflow® sensor. Alternatively, there may be pressure sensors 280 located both upstream and downstream of the pump 200, in order to measure a pressure difference across the pump 200. Preferably the pressure sensors 280 are piezoelectric sensors that measure the force applied to the tube. In this way, the pressure sensors 280 do not need to be integrated into the tube 150. Based on the measured pressure at the one or more pressure sensors 280, the drive signal to the drive mechanism may be altered to counteract any potential flow or pressure pulsations.

As will now be described, the drive mechanism for moving the pressing elements 220 between the first and second position may be embodied in a number of ways. Similarly, the actuator mechanism for moving the jaws 201, 202 between the open and the closed configuration may also be embodied in a number of ways.

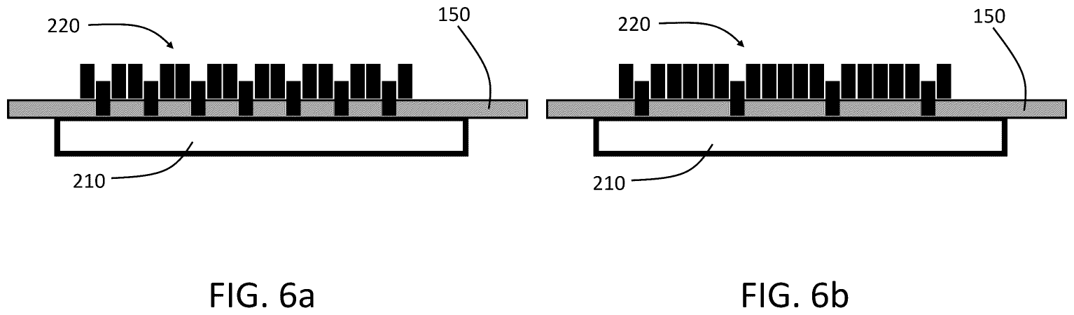

For example, the drive mechanism may be provided by a plurality of individual actuators to move each of the pressing elements 220 independently. Each of these individual actuators may be a piezo or electromagnetic actuator, for example. This arrangement allows miniaturization of the pump 200 and enables precise control of the flow profile over a range of flow rates. Furthermore, when using independently actuated pressing elements 220, the open configuration of the jaws 201, 202 may be reached simply by moving all of the pressing elements 220 away from the support plate 210. In this way, the actuator mechanism for moving the jaws 201, 202 between the open and the closed configuration may also be provided by the individual actuators. A tube sensor, or a sensor 280 such as the flow or pressure sensor 280 described above may also be used to address volume accuracy and smooth out pulsatile flow and compensate for variations in mechanical properties of the tube 150 with temperature and rate of operation. Additionally, as shown in FIGS. 6a and 6b, by making the pressing elements 320 independently addressable, smaller volumes of fluid may be separated and controlled by varying the distance between the pressing elements 320 that are in the first position. In the example shown in FIG. 6a, every third pressing element 320 is in the first position, thereby providing a plurality (e.g. a large number) of small volumes of fluid within the pump 300. In FIG. 6b, every sixth pressing element 320 is in the first position, thereby providing a smaller number of large volumes of fluid within the pump 300.

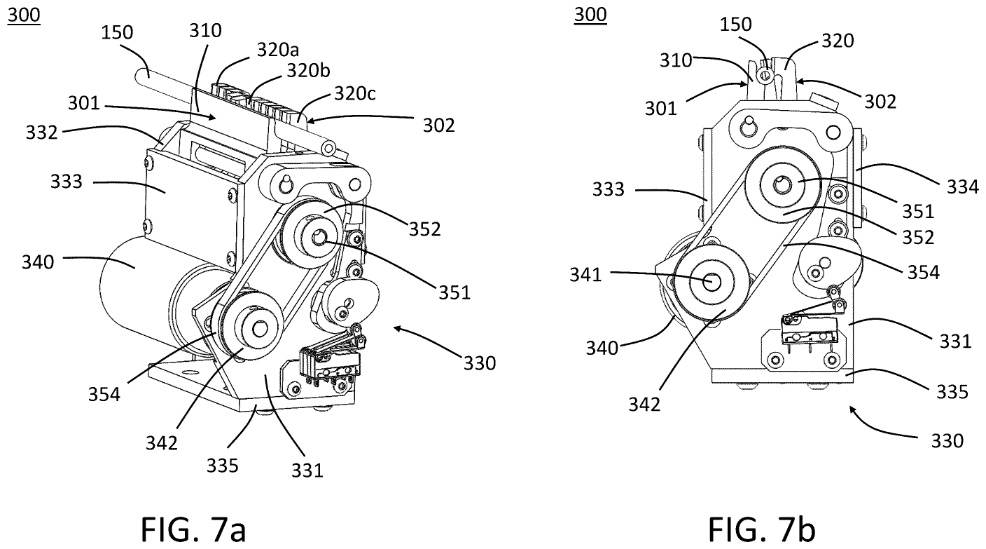

An embodiment of a peristaltic pump 300 will now be described with reference to FIGS. 7a to 7f. As shown in FIGS. 7a and 7b, the pump 300 has a first jaw 301 and a second jaw 302, together defining or forming a pair of jaws. The first jaw 301 is provided by a support plate 310 described above. The second jaw 302 is provided by a plurality of individual pressing elements 320 (only some of which are labelled for clarity). As shown, a tube 150 is located between the first jaw 301 and the second jaw 302.

The pump 300 also has a main body 330 comprising a front wall 331, a back wall 332, a first side wall 333, a second side wall 334, and a base 335. In order to move the pressing elements 320, the pump 300 comprises a drive mechanism, which may be contained at least partially within the body 330. The drive mechanism may be an electric motor 340 that drives rotation of a driveshaft 341. Preferably the motor 340 is controllable to maintain a constant rotation speed, even when experiencing variations in resistance from the tube 150 during use, so as to minimise pulsatile flow of the fluid. The motor 340 may be an electric stepper motor 340 or may be servo driven so that the speed and direction of the motor 340 may be precisely controlled.

Rotation of the driveshaft 341 is transmitted to a camshaft 351. A plurality of cams 350 (not shown) are attached to the camshaft 351, where each of the cams 350 corresponds to (and hence drives movement of) one of the pressing elements 330. In order to transmit rotation of the driveshaft 341 to the camshaft 351, the driveshaft 341 may be connected to a drivewheel 342, and the camshaft 351 may be connected to a camwheel 352. Rotation of the drivewheel 342 may be transmitted to the camwheel 352 by a cambelt 354. Alternatively, one or more gears may be used to transmit rotation of the driveshaft 341 to rotation of the camshaft 351. As a further alternative, the motor 340 may be connected directly to the camshaft 351.

FIG. 7c shows the pump 300 with certain components, such as the sidewalls 333, 334, the drivewheel 342 and the camwheel 352, been removed. A tube 150 is shown located between the support plate 310 and the pressing elements 320, i.e. the tube 150 is retained with the jaws 301, 302 defined by the support plate 310 and pressing elements 320, respectively. Each of the pressing elements 320 is in contact with a profiled cam 350. The pressing elements 320 are mounted to the pump 300 via a pivot shaft 325a that extends the length of the pump 300, and about which the pressing elements 320 are able to pivot when driven by their respective cam 350.

FIGS. 7d and 7e depict one of the pressing elements 320 and its cam 350 at different times, T₁, T₂, during use of the pump 300. The pressing element 320 has a tube contact portion 322 arranged to contact and compress the tube 150, a cam contact portion 324 that remains in contact with the cam 350, and a pivot 325 about which the pressing element 320 may rotate on the pivot shaft 325a. In order to maintain contact between each cam 350 and its corresponding pressing element 320, each pressing element 320 may have a weighted portion 326 to bias it towards the cam 350, as shown in FIG. 7c. Alternatively, a magnet or a resiliently deformable element such as a spring may bias the cam contact portion 324 of the pressing element 320 towards the cam 350.

In FIG. 7d, the pressing element 320 is shown in the first position at time T₁, where the profile of the cam 350 has pushed the cam contact portion 324 of the pressing element 320 away from the camshaft 351, thereby pivoting the pressing element 320 about its pivot 325 to move the tube contact portion 322 of the pressing element 320 towards the support plate 310 (and hence compress the tube). In FIG. 7e, the pressing element 320 is shown in the second position at time T₂. The cam 350 has been rotated by the camshaft 351 so that the cam contact portion 324 of the pressing element 320 is now closer to the camshaft 351. The pressing element 320 is thereby rotated about its pivot 325 to move the tube contact portion 324 away from the support plate 310 (and hence reduce compression of the tube 150). Therefore, during continued rotation of the cam 350, the pressing element 320 periodically moves between the first and second position. The profile of the cam 350 may define a range of motion of the pressing element 320 between the first and the second position. For example, the cam 350 may be profiled so that the pressing element 320 spends more time closer to the second position than the first position. Alternatively, the motion of the pressing element 320 may follow a substantially sinusoidal profile. A further example of a pumping profile will be described in relation to FIG. 14.

Referring again to FIG. 7c, each of the profiled cams 350 is attached to the camshaft 351 with a rotational offset relative to the cams 350 immediately on either side. This means that adjacent pressing elements 320 have a positional offset to each other, such that when the camshaft 351 is rotated, the pressing elements 320 make a compression wave to pump fluid through the tube. The speed of the motor 340 may be adjusted to control the flow rate of the fluid through the tube 150. Additionally, the direction of rotation of the motor 340 may be adjusted to reverse the flow direction.

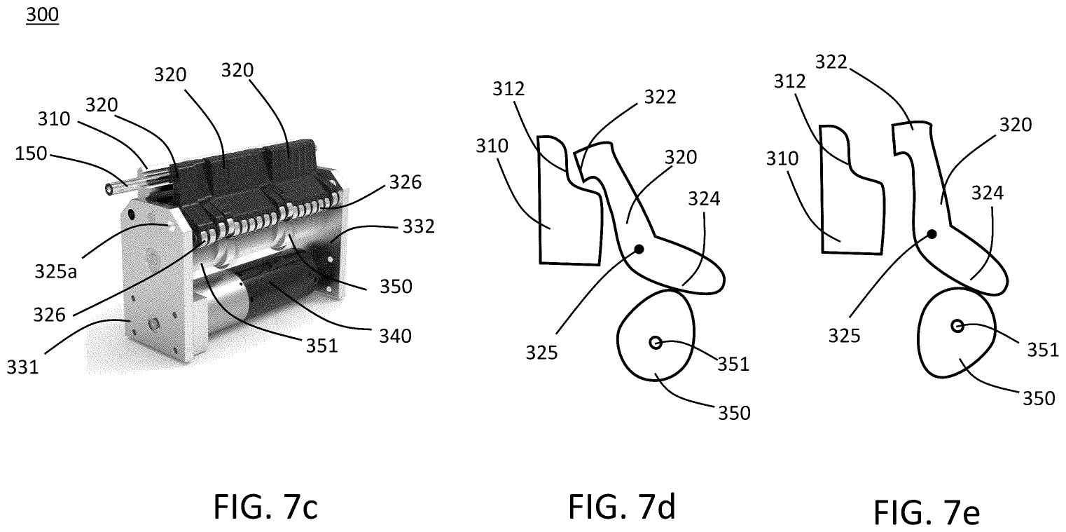

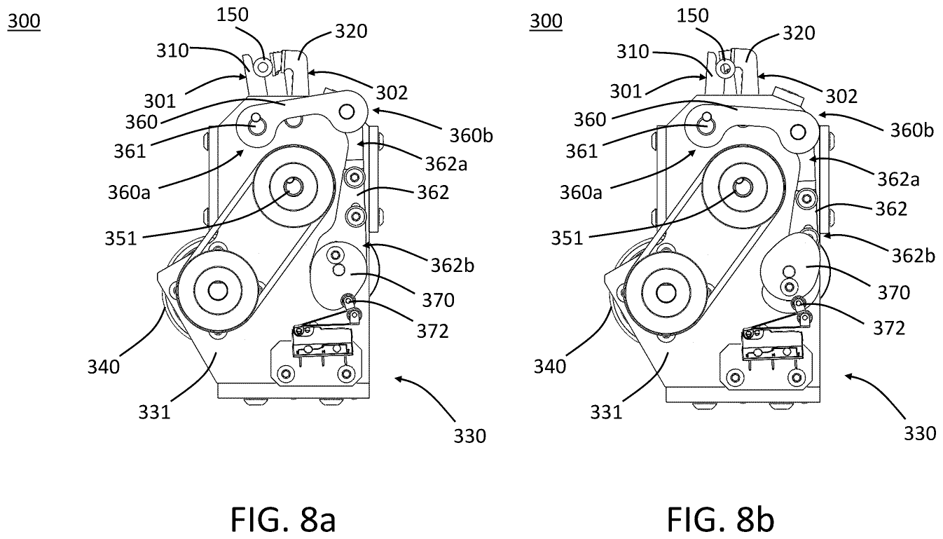

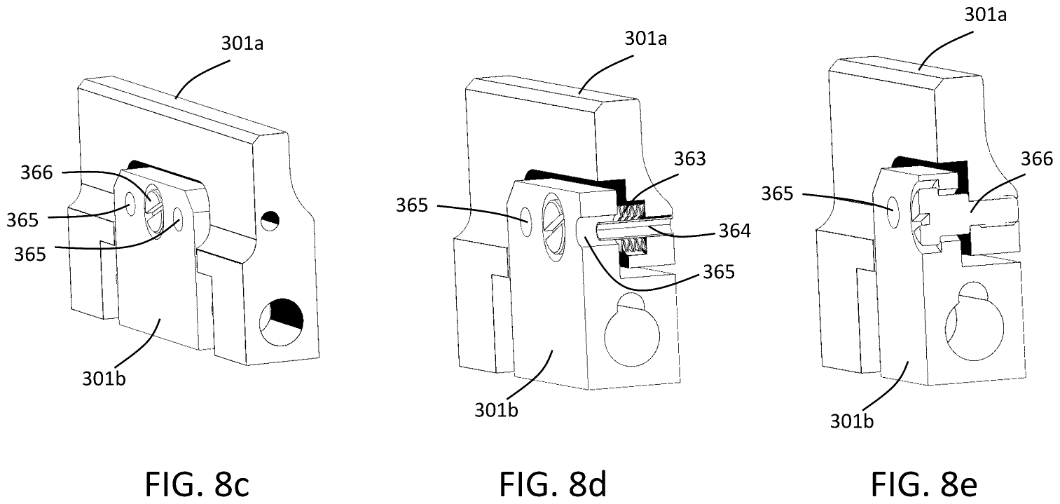

As will now be described with reference to FIGS. 8a and 8b, the first jaw 301 may be moved away from the second jaw 302 so that the tube 150 may be received and/or located therebetween. FIG. 8a depicts the pump 300 in an open configuration. In this open configuration, the first jaw 301 and the second jaw 302 are moved away from each other so that the tube 150 may be located between the jaws 301, 302. In particular, none of the pressing elements 320 of the second jaw 302 press the tube 150 against the support plate 310 when the jaws are in the open configuration.

FIG. 8b, depicts the pump 300 in a closed configuration. In this configuration, the first jaw 301 is moved towards the second jaw 302 so that the tube 150 is compressed between the jaws 301, 302. In the closed configuration, the pump 300 may operate as previously described, where sequential motion of the pressing elements 320 relative to the support plate 310 produces a compression wave along the tube 150 thereby pumping fluid through the tube 150. To move between the open configuration and the closed configuration, either or both of the components defining the first jaw 301 and the second jaw 302 may be moved by an actuator mechanism.

The actuator mechanism that moves the first jaw 301 relative to the second jaw 302 will now be described. As shown in FIGS. 8a and 8b, the first jaw 301 is connected to a linkage arm 360 on the exterior of the front wall 331. A first end 360a of the linkage arm 360 is connected to a shaft 361 which extends through the front wall 331 and the back wall 332 via a bearing (not shown). Rotation of the linkage 360 and the shaft 361 corresponds to movement of the first jaw 301 between the open and closed configuration. A second end 360b of the linkage arm 360 is pivotally connected to a first end 362a of an actuator arm 362. The actuator arm 362 is substantially perpendicular to the linkage arm 360 such that linear motion of the actuator arm 362 corresponds to rotational motion of the linkage arm 360. Linear motion of the actuator arm 362 may also be actuated in a number of ways, such as by using a linear actuator or a piston. In this embodiment, a camming element 370 is pivotally attached to a second end 362b of the actuator arm 362. The camming element 370 abuts against a block 372, such that rotation of the camming element 370 pushes the second end of the arm 362b further or closer to the block 372, thereby effecting substantially linear motion of the actuator arm 362. Rotation of the camming element 370 is produced by a motor (not shown), which may be located on the actuator arm 362. A corresponding/similar second linkage arm, a second actuator arm, a second camming element, a second block, and a second motor (not shown) may also be present on the exterior of the back wall 332. These corresponding components may move simultaneously to those on the front wall 331.

Movement of the first jaw 301 may be actuated in other ways. For example, a camming element may directly abut the second end 360b of the linkage arm 360 such that rotation of the camming element leads to reciprocating rotation of the linkage arm 360. Alternatively, a motor may be directly coupled to the shaft 361 so as to rotate the first jaw 301 between the open and closed configuration. While in this embodiment the first jaw 301 is moved by the actuator mechanism, the actuator mechanism may instead move the second jaw 302, or it may move both jaws 301, 302 simultaneously.

The first jaw 301 and second jaw 302 may extend beyond the main body 330 of the pump 300, so that the tube 150 may be easily received/located and subsequently removed from between the jaws 301, 302 without being obstructed by other parts of the pump 300. As well as providing ease of access to the tube 150, this arrangement can minimise the length of tube required to interact with the pump 300. In this way, the tube 150 can easily be located between the jaws 301, 302 autonomously, such as by a robotic gripping mechanism (not shown). Alternatively, the pump 300 may be configured as a robotic end effector 100 so that the pump 300 may be moved until the tube 150 is located between the first jaw 301 and the second jaw 302. The robotic end effector 100 may also contain a machine vision system to detect when the tube 150 has successfully been located between the jaws 301, 302 of the pump. The machine vision system may be used as well as, or instead of, the tube sensors previously described above, for example to locate and pick up the tube 150.

A controller (not shown) may be located in the body 330 of the pump 300 or may be located elsewhere in the automated system 2. The controller is used to control a sequence of operation of the pump 300 such as to actuate the drive mechanism and the actuator mechanism. For example, the controller may move the jaws 301, 302 into the open configuration so that a tube may be located therebetween. Subsequently, the controller may move the jaws 301, 302 from the open configuration into the closed configuration when the machine vision system and/or the tube sensors determine that the tube 150 is correctly located between the jaws 301, 302.

Alternatively, or additionally, the controller may use the robotic device 3 to move the pump 300 until the tube 150 is located between the jaws 301, 302, and then subsequently move the jaws 301, 302 into the closed configuration. The controller may also receive instructions from an operator to move the jaws 301, 302 between the open and closed configurations. Once the tube 150 is correctly engaged by the jaws 301, 302 in the closed configuration, the controller activates the drive mechanism so as to being a pumping operation.

The pump 300 may comprise a power supply (not shown) and/or a transformer in order to power components such as the drive mechanism and the actuation mechanism. The power supply and/or transformer may be located at least partially in the main body 330 of the pump 300. Alternatively, the power supply and/or transformer may be located external to the pump 300 and wires may be used to transmit electrical power to the pump 300. For example, where the pump 300 is located on the end effector 100, the power supply may be located elsewhere in the automated system 2 or the robotic device 3, in order to reduce the size and weight of the end effector 100.

As shown in FIGS. 8c to 8e, the first jaw 301 may comprise a first part 301a which is resiliently coupled to a second part 301b by a spring element 363 (or an equivalent resilient means). In this example, the spring element 363 comprises two Belleville washer stacks 363 (only one visible in the sectional view in FIG. 8d). A pin 364 extends from the first part 301a of the first jaw 301, through the Belleville washer stack 363, and into a bore 365 in the second part 301b of the first jaw 301a, thereby maintaining alignment of the Belleville washer stack 363. A limit screw 366 connects the first part 301a of the first jaw 301 to the second part 301b of the first jaw 301, in order to restrict the range of motion.

The jaw 301 is thereby moved to the closed configuration by the application of force via the spring element 363, which allows a degree of compliance between the first and second parts 301a, 301b of the first jaw 301. In this way, the pump 300 is able to accommodate small variations in materials and dimensions of the tube 150, in particular when the jaws 301, 302 are in the closed configuration. Compliance of the first jaw 301 may be achieved in other ways, such as by forming at least part of the first jaw 301 from a resiliently deformable material. Alternatively, the second jaw 302 may be resiliently deformable.

A load cell (not shown) arranged in series with the spring element 363 may also be used to measure the actual clamp force being exerted and also to infer the point at which the tube 150 is pinched shut by the pressing element from the load displacement profile. For example, the force measured by the load cell may increase sharply once the tube becomes fully pinched shut. In conjunction with a controller, the use of a load cell may enable automatic clamp adjustment for a range of tube geometries as well as in response to any creep or permanent set in the tubing during pumping which could affect metering performance.

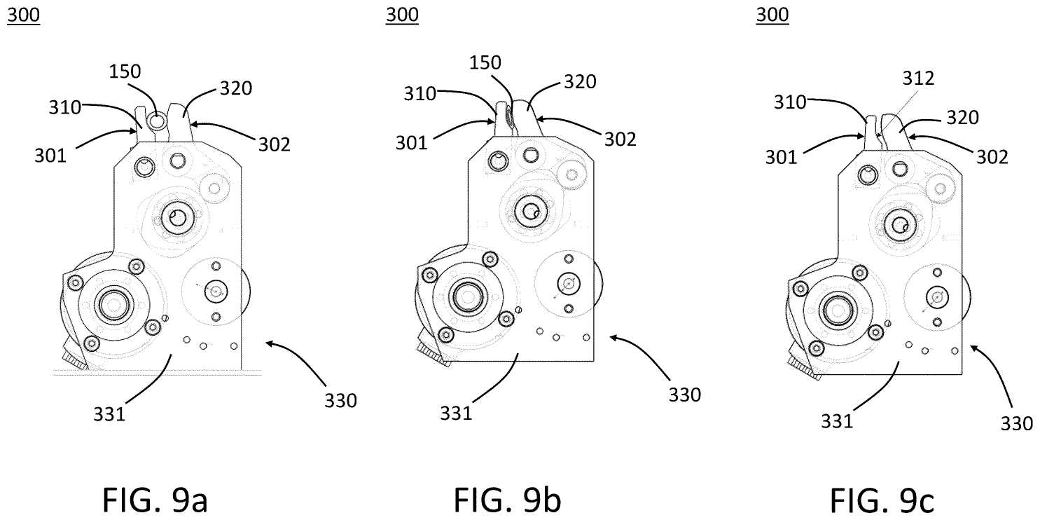

FIGS. 9a to 9c depict a side view of the pump 300 where several components have been hidden for clarity. In particular, the linkage arm 360, the actuator arm 362, the drivewheel 342, the camwheel 352, and the cambelt 354 are not shown. Furthermore, only the pressing element 320 that is closest to the front wall 331 is visible. In FIG. 9a, the jaws 301, 302 are shown in the open configuration, and a tube 150 is positioned between the jaws 301, 302. In FIG. 9b, the actuator mechanism has moved the jaws 301, 302 into the closed configuration, so that the tube is retained between the jaws 301, 302. Additionally, due to rotation of the camshaft 351, the pressing element 320 is now in the first position where the tube 150 is compressed between the pressing element 320 and the support plate 310. FIG. 9c depicts the pump 300 in the same position as in FIG. 9b, but where the tube 150 has been removed.

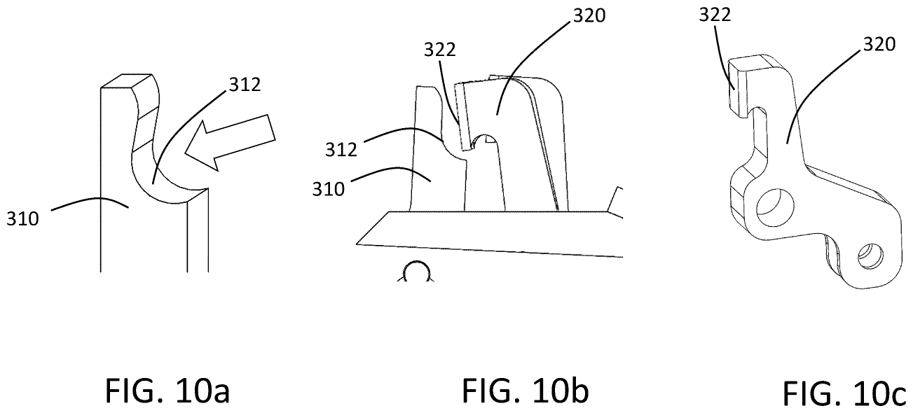

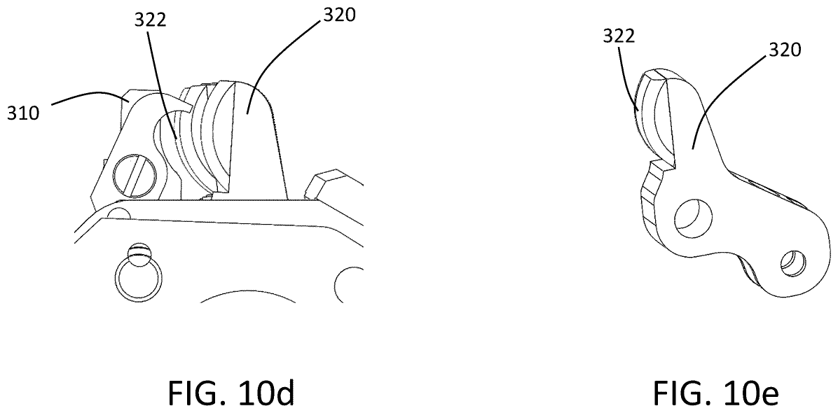

The support plate 310 may have a profiled portion 312 to ensure that when the jaws 301, 302 are moved from the open to the closed configuration, the tube 150 is urged towards a predetermined position between the jaws 301, 302. In this way, the tube 150 is automatically aligned between the jaws 301, 302 in a consistently repeatable position without need for any operator intervention or any additional alignment steps. During a pumping operation, the profiled portion 312 also keeps the tube 150 aligned between the jaws 301, 302 defined by the support plate 310 and pressing elements 320, respectively, and prevents the tube 150 from being forced out of the pump 300. With particular reference to FIGS. 10a to 10c, when the pressing elements 320 compress the tube 150 against the support plate 310 (in the direction of the arrow), they do so in a direction that urges the tube 150 towards a predetermined position on the profiled portion 312 of the support plate 310. For example, the profiled portion 312 may be a curved portion, which may have a radius of curvature that corresponds to the tube 150. Alternatively, the profiled portion 312 may be a corner or indentation in the support plate 310 into which the tube 150 is pressed by the pressing elements 320. The tube contact portion 322 of the pressing elements 320 may have a planar profile so that the tube 150 is substantially enclosed between the curved profile of the support plate 310 and the planar profile of the pressing elements 320. In this way, the tube 150 is always retained along a predetermined axis during use of the pump 300, thereby allowing consistent and reliable operation of the pump 300.

Alternatively or additionally, the tube contact portion 322 of the pressing elements 320 may also have a profiled portion to urge the tube 150 into a predetermined position. With particular reference to FIGS. 10d and 10e, the tube contact portion 322 of the pressing element 320 has a curved profile that corresponds to the curved profile of the support plate 310. In this way, the tube 150 is more uniformly compressed between the support plate 310 and the pressing element 320, thereby optimising tube occlusion.

Additionally, either or both of the profiled portion 312 and/or the tube contact portion 322 may have a high friction coefficient to prevent the movement of the tube 150 from its predetermined position. This may be achieved by using materials with a high friction coefficient, and/or may be achieved by using a ridged or textured surface. Alternatively, either or both of the profiled portion 312 and/or the tube contact portion 322 may be made from materials with a low friction coefficient, so as to ensure the tube 150 can slide into the predetermined position on the profiled portion 312. This may be achieved by using materials with a low friction coefficient, and/or may be achieved by using a smooth surface.

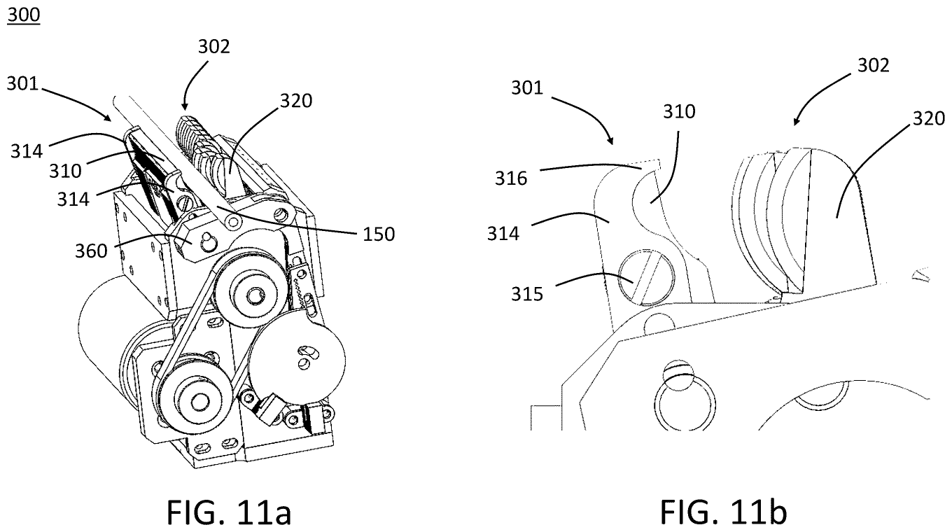

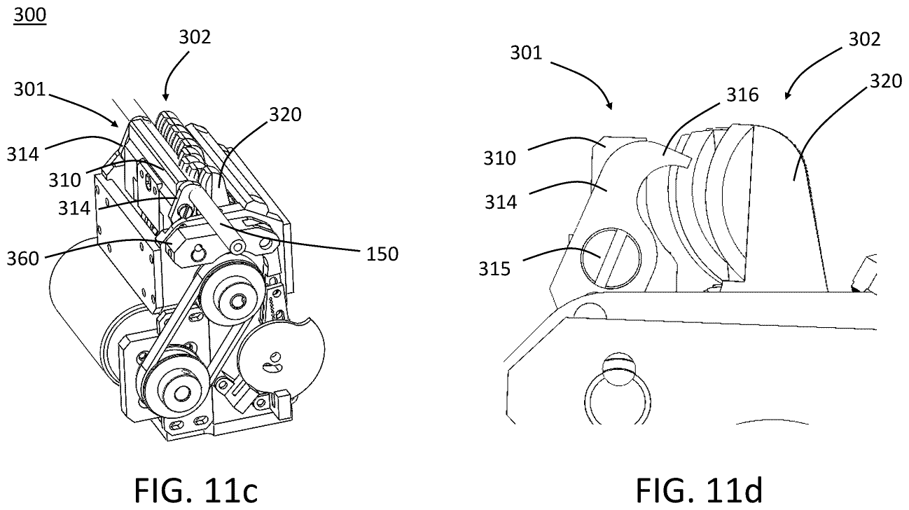

As will now be described with reference to FIGS. 11a to 11d, the pump 300 may also comprise a tube latch 314 on each side of the support plate 310. The tube latches 314 have a pivot 315 and a hooked portion 316. In FIGS. 11a and 11b, the jaws 301, 302 are in the open configuration where the support plate 310 is moved away from the pressing elements 320. The tube latches 314 are in a retracted position so that the tube 150 may be easily located between the jaws 301, 302.

In FIGS. 11c and 11d, the jaws 301, 302 are in the closed configuration, where the support plate 310 is moved towards the pressing elements 320. The tube latches 314 have rotated about their pivot 315 into an engaged position, where the hooked portion 316 of the tube latch 314 holds the tube 150 between the jaws 301, 302. The tube latches 314 are moved between the retracted and the engaged position by the actuator mechanism described above. In particular, the tube latches 314 are coupled to the linkage arm 360, so that rotation of the linkage arm 360 moves the tube latches 314 between the retracted and the engaged position. While FIGS. 11a to 11d depict the tube latches 314 used on a pump 300 where the contact portion 322 of the pressing elements 320 have a curved profile (see FIGS. 10d and 10e), the tube latches 314 may also be used on a pump 300 where the contact portion 322 of the pressing elements 320 has a planar profile (see FIGS. 10a to 10c). Furthermore, while the tube latches 314 are depicted as being on either side of the support plate 310, they may equivalently be located on either side of the plurality of pressing elements 320.

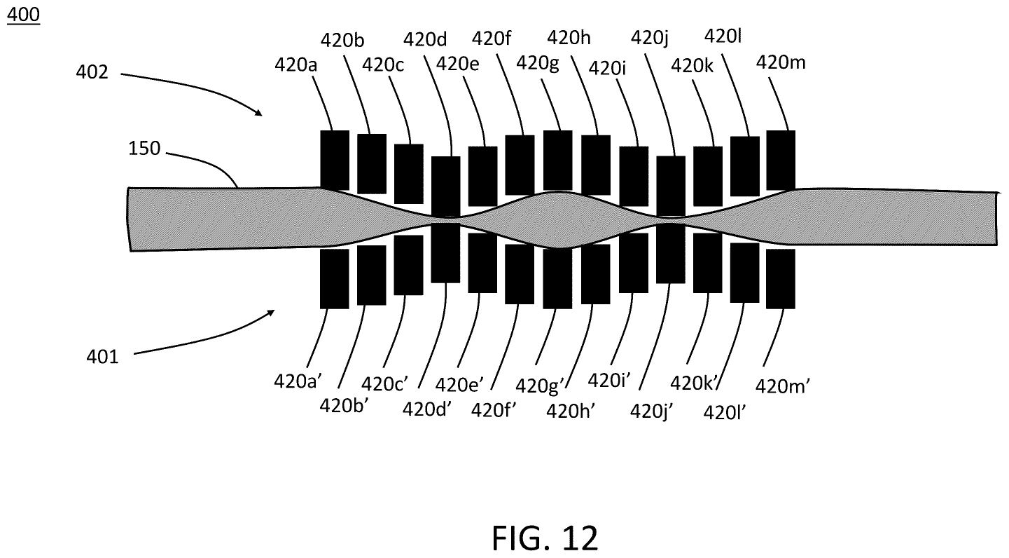

FIG. 12 depicts another embodiment of a pump 400 according to the present invention. The pump 400 is similar to the pump 200, 300 of the above-described embodiments, wherein the second jaw 402 is provided by a plurality of pressing elements 420. However, in this embodiment, the first jaw 401 is also provided by a (second) plurality of pressing elements 420′ that oppose each of the pressing elements 420 on the second jaw 402. The motion of the plurality of pressing elements 420′ on the first jaw 401 mirrors the motion of the plurality of pressing elements 420 on the second jaw 402, such that the sequential compression of the tube 150 between opposing pressing elements 420, 420′ produces a compression wave that pumps fluid along the tube 150 in a similar manner as already described. Gripping mechanisms, such as the gripping mechanisms 290, 292 described in relation to FIGS. 4a and 4b may also be included in this embodiment. A flow sensor, such as the flow sensor 280 described in relation to FIG. 5, may also be used with this embodiment.

The second plurality of pressing elements 420′ on the first jaw 401 may be driven in a similar way to as described above for the first plurality of pressing elements 420. For example, the motor 440 may be connected to a separate camshaft (not shown) to move each of the pressing elements 420′. Alternatively, a single camshaft may be arranged to drive the pressing elements 420, 420′ on both of the first and second jaws 401, 402. As a further alternative, the pressing elements 420′ may be independently actuated using piezo or electromagnetic actuators, as described previously with reference to FIGS. 6a and 6b. One or both of the pluralities of pressing elements 420, 420′ may have a profiled portion similar to those described above, in order to automatically align and retain the tube 150 between the jaws 401, 402 both during a pumping operation and during closing of the jaws 401, 402. Either or both of the pluralities of pressing elements 420, 420′ may be resiliently deformable so as to allow for small variations in tube materials and dimensions.

As such, the arrangement of pressing elements 420, 420′ in this embodiment provides a means for effecting a peristaltic pumping action on a fluid contained within the tube 150 when the tube 150 is retained between the pair of jaws 401, 402 in the closed configuration.

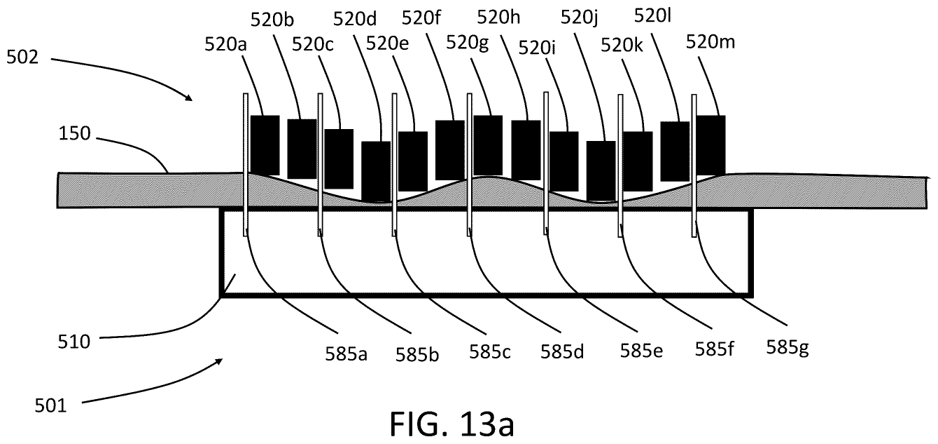

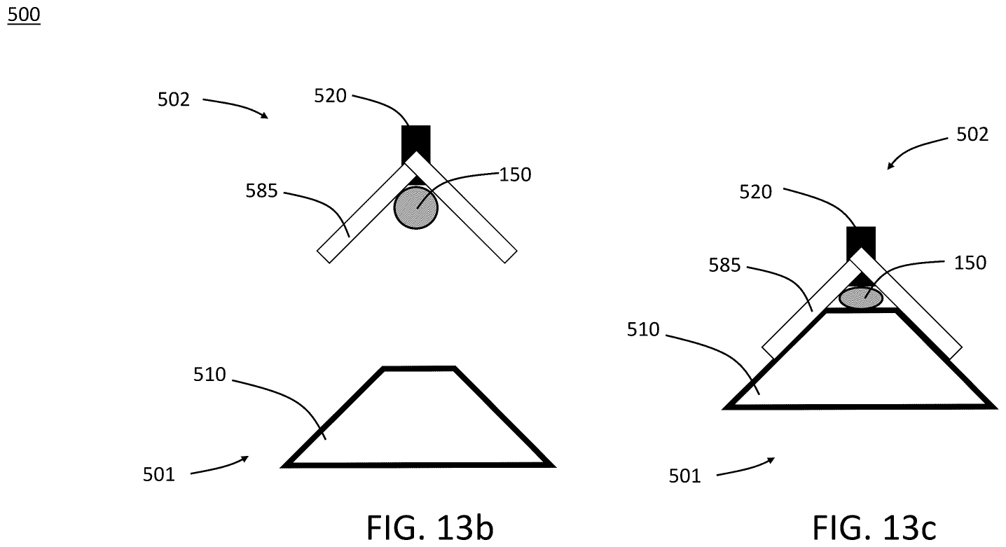

FIGS. 13a to 13c depict another embodiment of a pump 500 according to the present invention. The pump 500 is similar to the pumps 200, 300, 400 already described above. In FIG. 13a, the jaws 501, 502 are shown in the closed configuration. In this embodiment, a plurality of tube retaining elements 585a-585g (which may also be referred to as “tube clips”) are located between the pressing elements 520. As depicted in FIG. 13a, two pressing elements 520 are present between each pair of tube retaining elements 585. However, more or fewer tube retaining elements 585 may be present; for example, there may be only one pressing element 520 between adjacent tube retaining elements 585, or there may be more than two pressing elements 520 between adjacent tube retaining elements 585.

FIGS. 13b and 13c depict a side view of the pump 500 shown in FIG. 13a. In FIG. 13b, the jaws 501, 502 are in the open configuration, and in FIG. 13c, the jaws 501, 502 are in the closed configuration. As shown in FIG. 13c, the tube retaining elements 585 are arranged to interlock (or “mate”) with a profiled portion 512 on the support plate 510 when the jaws 501, 502 are in the closed configuration. In this way, the tube 150 is completely surrounded by the combination of the tube retaining elements 585 and the support plate 510 so that the tube 150 is retained in a predetermined position during a pumping operation. One or more of the tube retaining elements 585 may comprise a high friction material for similar reasons to the gripping mechanisms 290, 292 described previously. It will be appreciated that gripping mechanisms 290, 292 may be used in addition to the tube retaining elements 585.

In the example depicted in FIGS. 13b and 13c, the tube retaining elements 585 are substantially L-shaped and interlock with a trapezium shaped support plate 510. However, other shapes of both the tube retaining elements 585 and support plate 510 may be used. It will be appreciated that the tube retaining elements 585 may also be incorporated into any of the embodiments described. For example, the pressing elements 520 may be independently actuated by piezo or electromagnetic actuators or may be actuated with a motor and cams similar to those described in relation to FIGS. 7a to 7f. Alternatively, or additionally, the first jaw 501 may be provided by a plurality of opposing pressing elements 520′, similar to the embodiment described in relation to FIG. 12.

A predetermined sequence of the pressing elements may be controlled in order to provide a particular “pumping profile”. FIG. 14 depicts an embodiment of a pump 600 where the pumping profile is asymmetric. In this example, the pressing elements 620 initially compress the tube 150 quickly or steeply, before gradually releasing the tube 150 over a greater length of the tube 150. In other words, the pressing elements 620 upstream of the pinched (occluded) portion compress the tube 150 more steeply (along a first portion of tube 150), while those downstream allow the tube 150 to recover more gradually (along a second portion of tube 150, which is longer than said first portion of the tube). For example, the angle between pressing elements 620 upstream of the occluded portion may be about 60 degrees. Immediately downstream of the occluded portion, the angle between adjacent pressing elements may initially be about 60 degrees before gradually decreasing to 30 degrees. The position of the pressing elements 620 may be inversely proportional to the distance from the occluded portion, leading to a gradually decreasing angle. The profile downstream of the occluded portion may be calculated based on the Flamant solution.

In this way, the wear to the tube 150 may be reduced by lowering shear forces that may be applied tangentially to the surface of the tube 150. Furthermore, the asymmetric profile may account for the pressure difference from before and after the pinched portion, thereby reducing deformation of the tube 150. While a particular pumping profile is shown in FIG. 14, it will be appreciated that other profiles may be provided. The pumping profile may be selected based on the tube 150 properties (e.g., outer diameter, thickness, material) and/or based on the fluid being pumped through the tube 150. The pumping profile may be determined using computer modelling and/or by experiment.

Where the drive mechanism comprises a plurality of cams 350 (e.g. see FIGS. 7a to 7e), the cams 350 may have an asymmetrical profile, whereby constant rotation of the camshaft 351 moves the pressing elements 320 at different speeds at different times throughout the cycle. For example, a rapid increase in radius of a cam 350 results in a faster movement of its corresponding pressing element 320. In addition, the rotational offset between adjacent cams 320 may vary across the length of the camshaft 351 in order to provide asymmetric profiles. Furthermore, the cams 350 need not all have the same shape; cams 350 near the edges of the pump may have a different shape to those near the centre.

Where the drive mechanism is provided by a plurality of individual (e.g. linear) actuators that move each of the pressing elements independently (see FIGS. 6a and 6b), the actuators may be programmed or controlled to move with specific timings and/or speeds to provide the desired pumping profile, such as an asymmetric pumping profile.

While the foregoing is directed to exemplary embodiments of the present invention, it will be understood that the present invention is described herein purely by way of example, and modifications of detail can be made within the scope of the invention. Furthermore, one skilled in the art will understand that present invention may not be limited to the embodiments disclosed herein, or to any details shown in the accompanying Figures that are not described in detail herein or defined in the claims. Indeed, such superfluous features may be removed from the Figures without prejudice to the present invention. It will also be appreciated that particular combinations of the various features described and defined in any aspects described herein can be implemented and/or supplied and/or used independently. Any apparatus feature described herein may also be incorporated as a method feature, and vice versa.

Moreover, other and further embodiments of the invention will be apparent to those skilled in the art from consideration of the specification, and may be devised without departing from the basic scope thereof, which is determined by the claims that follow.

1. A linear peristaltic pump for a bioprocessing system, comprising:
at least one pair of jaws formed by a first jaw and a second jaw, the first jaw being moveable relative to the second jaw such that the pair of jaws can be moved between an open configuration in which the jaws are spaced apart for receiving a flexible tube therebetween and a closed configuration in which the jaws are brought together so as to retain the tube therebetween;
an actuator mechanism configured to control movement of the first jaw relative to the second jaw; and
means for effecting a peristaltic pumping action on a fluid contained within the tube when the tube is retained between the pair of jaws in the closed configuration.

2. The linear peristaltic pump of claim 1, wherein the means for effecting a peristaltic pumping action comprises:
a support portion configured to support the tube; and
a plurality of pressing elements, each pressing element being moveable relative to the support portion between a first position, in which the pressing element compresses a portion of tube supported by the support member whereby to restrict the flow of fluid through said portion, and a second position, in which said portion of tube is not substantially compressed and the flow of fluid is not restricted.

3. The linear peristaltic pump of claim 2, wherein at least one of the pressing elements is configured always to be in contact with a tube retained between the jaws when in the closed configuration.

4. The linear peristaltic pump of claim 2, further comprising a drive mechanism for moving the pressing elements individually relative to the support portion so as to effect a peristaltic pumping action on a fluid contained within a tube retained between the jaws.

5. (canceled)

6. The linear peristaltic pump of claim 1, further comprising a detector to determine whether the tube is received between the jaws.

7. (canceled)

8. The linear peristaltic pump of claim 4, further comprising a main body that houses the drive mechanism and/or the actuator mechanism, wherein said pair of jaws extends beyond or outside the main body.

9. The linear peristaltic pump of claim 1, wherein at least one of the first jaw and the second jaw comprises a profiled portion, such that when the pair of jaws is moved into the closed configuration the flexible tube is urged towards a predetermined position within the profiled portion.

10. The linear peristaltic pump of claim 9, wherein the profiled portion of the jaw comprises a curved portion of the jaw, preferably having a radius that generally corresponds to the radius of the tube to be received thereby.

11. The linear peristaltic pump of claim 1, further comprising one or more gripping mechanisms arranged to engage the tube, wherein:
gripping mechanism is configured to load and/or unload the tube from the pair of jaws when the jaws are in the open configuration; and/or
wherein the gripping mechanism is configured to retain the tube so as to inhibit longitudinal motion of the tube through the pair of jaws during a pumping operation.

12. (canceled)

13. (canceled)

14. The linear peristaltic pump of claim 11, wherein a portion of the gripping mechanism arranged to contact the tube comprises a material with higher friction than the material on a portion of the pair of jaws arranged to contact the tube.

15. (canceled)

16. The linear peristaltic pump of claim 2, wherein the support portion is arranged to define the first jaw, and the plurality of pressing elements are arranged to define the second jaw.

17. (canceled)

18. The linear peristaltic pump of claim 16, wherein the support portion is defined by a further plurality of pressing elements movable to oppose said plurality of pressing elements of the second jaw.

19. The linear peristaltic pump of claim 1, further comprising a flow sensor configured to measure the flow of fluid through a tube secured between the jaws.

20. (canceled)

21. The linear peristaltic pump of claim 1, further comprising a pressure sensor configured to measure the pressure of fluid in a tube secured between the jaws.

22. (canceled)

23. The linear peristaltic pump of claim 1, wherein the means for effecting a peristaltic pumping action is configured to effect an asymmetric peristaltic pumping action along a length of the tube.

24. The linear peristaltic pump of claim 23, wherein the asymmetric peristaltic pumping action is configured to pinch a portion of the tube quicker than it releases said pinched portion.

25. The linear peristaltic pump of claim 1, wherein control of the pump is automated.

26. The linear peristaltic pump of claim 25, wherein the actuator mechanism is configured to receive signals from a controller to move the jaws between the open and closed configurations, wherein the controller is further configured to control the means for effecting a peristaltic pumping action to commence pumping once the jaws are in the closed configuration.

27. A robotic device comprising a robotic end effector, wherein the robotic end effector comprises a linear peristaltic pump, the linear peristaltic pump comprising:
at least one pair of jaws formed by a first jaw and a second jaw, the first jaw being moveable relative to the second jaw such that the pair of jaws can be moved between an open configuration in which the jaws are spaced apart for receiving a flexible tube therebetween and a closed configuration in which the jaws are brought together so as to retain the tube therebetween;
an actuator mechanism configured to control movement of the first jaw relative to the second jaw; and
means for effecting a peristaltic pumping action on a fluid contained within the tube when the tube is retained between the pair of jaws in the closed configuration.

28. (canceled)

29. A method of performing a peristaltic pumping action on a tube, comprising:
actuating a plurality of pressing elements independently to compress and release a portion of said tube so as to pump a fluid therethrough,
wherein said plurality of pressing elements are arranged in a substantially linear configuration.

30. (canceled)

31. (canceled)

32. (canceled)

33. (canceled)
